(19) United States
(12) Patent Application Publication
(10) Pub. No.: US 2026/0276632 A1
(43) Pub. Date: Sep. 17, 2026

(54) APPARATUS AND METHOD FOR THE CAPTURE OF INTRACELLUAR ACTIVITY WITH NANOFIDS

(71) Applicants: Bruce T. HOPE, Ellicott City, MD (US); Mark A. WELLS, San Diego, CA (US); Gregory D. SUTTON, Del Mar, CA (US)

(72) Inventors: Bruce T. HOPE, Ellicott City, MD (US); Mark A. WELLS, San Diego, CA (US); Gregory D. SUTTON, Del Mar, CA (US)

(21) Appl. No.: 19/665,300

(22) Filed: May 1, 2026

Related U.S. Application Data
(60) Provisional application No. 63/799,904, filed on May 5, 2025.

(51) Int. Cl. G01N 33/543; G01N 27/30; G01N 27/414

(52) U.S. Cl. CPC G01N 33/54373; G01N 27/308; G01N 27/4145

(57) ABSTRACT

An intracellular monitoring device including plural multilayer nanofid apparatus that fit completely inside plural living cells, and cause no significant impairment, to the plural cells normal biological processes. The plural nanofid apparatus monitor plural cells for its level of a biological molecules (e.g., Ca⁺, etc.) of interest. If the biological molecules of interest reaches or exceeds a pre-determined threshold, the plural nanofid apparatus in the plural living cells transmits an electromagnetic signal, received by an optical collection system. Each nanofid has its electromagnetic signal encoded with a unique frequency. Detection of the frequency components, in the signals received by the optical collection system with a signal processing method, permits identification of the source nanofid apparatus in plural individual living cells at the individual cell level.

### CROSS REFERENCES TO RELATED APPLICATIONS

The utility patent application claims priority to U.S. Provisional patent application No. 63/799,904, filed on May 5, 2025, the contents of which are incorporated by reference.

### FIELD OF INVENTION

The present invention relates generally to capturing cellular activity, and, more particularly, to capturing intra-cellular activity with a monitoring device that will be implanted in each cell to be monitored.

### BACKGROUND OF THE INVENTION

The chemical and electrical processes inside living cells are known to be extremely complicated, and existing techniques for collection of information on such processes, with sensing apparatus external to the cell, are known to have a variety of significant limitations.

One solution to the collection of cellular activities includes U.S. Pat. No. 11,627,907, that issued to Hope, et al. that teaches a system for intracellular monitoring including: “An intracellular monitoring device (IMD) that fits completely inside a living cell, and causes no significant impairment, to a cell's normal biological processes. The IMD monitors a cell for its level of a biological substance (e.g., calcium ion concentration) of interest. If the biological substance reaches or exceeds a threshold, the IMD transmits an electromagnetic signal, received by an antenna outside the cell. Each IMD has its electromagnetic signal encoded with a unique frequency. Detection of the frequency components, in the signals received by an antenna, permits identification of the source IMD's. A high calcium ion concentration is indicative of a strongly-activated cerebral cortex neuron. Brain tissue is relatively transparent to near infrared, making it a good frequency band, for the electromagnetic signals from neuron-monitoring IMD's. The near infrared of each IMD can be produced by quantum dots, powered by bioelectric catalysis triggered by high calcium ion concentration.”

Another solution related to the collection of cellular activities includes U.S. Pat. No. 12,268,512, that issued to Hope, et al. that issued to Hope, et al. that teaches a system for intracellular monitoring including: “an intracellular monitoring device (IMD) that fits completely inside a living cell, and causes no significant impairment, to a cell's normal biological processes. The IMD monitors a cell for its level of a biological substance (e.g., calcium ion concentration) of interest. If the biological substance reaches or exceeds a threshold, the IMD transmits an electromagnetic signal, received by an antenna outside the cell. Each IMD has its electromagnetic signal encoded with a unique frequency. Detection of the frequency components, in the signals received by an antenna, permits identification of the source IMD's. A high calcium ion concentration is indicative of a strongly-activated cerebral cortex neuron. Brain tissue is relatively transparent to near infrared, making it a good frequency band, for the electromagnetic signals from neuron-monitoring IMD's. The near infrared of each IMD can be produced by quantum dots, powered by bioelectric catalysis triggered by high calcium ion concentration.”

An example area, where new mechanisms for sensing intracellular activity are particularly desirable, is the sensing of intracellular calcium as a means for monitoring neural activity.

Each existing technique, for the capture of neural-activity, fits into one of the two following categories: (1). Captures the average activity of many neurons (hundreds of thousands) in a large cortical region, but the activity of the individual neurons within the monitored region is unknown. An example technique in this category is functional magnetic resonance imaging. Since specific thoughts and memories are encoded by highly specific patterns of sparsely distributed neurons called neuronal ensembles or engrams throughout the brain, capturing the average activity of many neurons in a large region cannot capture events relevant to encoding specific high-resolution thoughts or memories, (2) Captures the activity of individual neurons, but only a small number of adjacent neurons (e.g., on the order of 100-1000) can be simultaneously monitored. Example techniques in this category include electrode arrays and calcium imaging. The low number of neurons captured by these latter techniques can capture only low-resolution information, and not high resolution, highly specific thoughts or memories.

None of these solutions solve all of the problems associated with neuronal activity monitoring.

Silicon MEMS are used with shank electrodes (from Buzsaki) to capture the activity of 100-300 neurons per mm² of cortex or 0.3% coverage under optimal lab conditions (Shobe 2015, J. Neurophys.). Coverage, for example, refers to a number of cortical neurons that are recorded under a given area of cortical surface. For example, there are ~100,000 neurons under each mm² of cortical surface etc.

NEURALINK (Elon Musk) captures the activity of neurons via 3072 silicon electrodes per 425 mm² equal to 7.2 electrodes per mm² or ~0.007% neuron coverage.

SMART DUST (Maharbiz lab) (Seo et al 2016, Neuron) uses one surface electrode (LFP) on the neural surface to capture the activity of neurons with 1 electrode per mm² or ~0.001% neuron coverage. Ultrasonic power and backscatter are used to provide two-way information.

NEUROGRAINS (Nurmikko lab) (Lee et al 2021, Nature Electronics) captures the activity of neurons on one surface electrode (LFP) per 100 microns with 100 electrodes per mm² or ~0.1% neuron coverage. Radio Frequency (RF) is used to provide two-way information.

It would therefore be desirable to have neuronal-activity monitoring systems that capture both the activity of all neurons in a large area and, within that area, activity on an individual-neuron level.

It would therefore be desirable to have intracellular activity monitoring systems that capture both the activity of all cells in a large area and, within that area, activity on an individual-cell level.

### SUMMARY OF THE INVENTION

In accordance with preferred embodiments of the present invention, some of the problems associated with intracellular monitoring are overcome. An apparatus and method for the capture of intracellular activities with nanofids is presented.

An intracellular monitoring device including plural multilayer nanofid apparatus that fit completely inside plural living cells, and cause no significant impairment, to the plural cells normal biological processes. The plural nanofid apparatus monitor plural cells for its level of a biological intracellular molecules (e.g., Ca⁺, etc.) of interest. If the biological intracellular molecules of interest reaches or exceeds a threshold, the plural nanofid apparatus in the plural living cells transmits an electromagnetic signal, received by a nanofid optical collection system. Each nanofid has its electromagnetic signal encoded with a unique frequency. Detection of the frequency components, in the signals received by the nanofid optical collection system utilizes a signal processing system that permits identification of the source nanofid apparatus in plural individual living cells at the individual cell level.

The foregoing and other features and advantages of preferred embodiments of the present invention will be more readily apparent from the following detailed description. The detailed description proceeds with references to the accompanying drawings.

### BRIEF DESCRIPTION OF THE DRAWINGS

Preferred embodiments of the present invention are described with reference to the following drawings, wherein:

FIG. 1 is a block diagram illustrating an exemplary nanofid apparatus;

FIG. 2. is a block diagram illustrating an exemplary nanofid apparatus;

FIG. 3 is a block diagram illustrating an exemplary nanofid apparatus system;

FIG. 4 is a block diagram illustrating additional details of the exemplary nanofid apparatus;

FIG. 5 is a block diagram illustrating additional details of a Quantum Dot (QD) Light Emitting Diode (LED);

FIG. 6 is a block diagram illustrating example detailing of a pulse comparator of the exemplary nanofid apparatus;

FIG. 7 is a block diagram illustrating typical time course for intracellular calcium in an activated cell (e.g. neuron) and the function of a pulse comparator in the exemplary nanofid apparatus;

FIG. 8 is a block diagram illustrating a signal processing system including pulse comparator timing diagram of the exemplary nanofid apparatus;

FIG. 9 is a block diagram illustrating additional details of an exemplary nanofid apparatus strobing method;

FIG. 10 is a block diagram illustrating light coupling into a nanofid optical collection system;

FIG. 11 is a block diagram illustrating additional details of an exemplary nanofid apparatus data flow;

FIG. 12 is a block diagram illustrating an exemplary nanofid apparatus inserted in a human cell;

FIG. 13 is a flow diagram illustrating a method for determining intracellular activity with nanofid apparatus;

FIG. 14 is a flow diagram illustrating a method for processing intracellular activity with nanofid apparatus;

FIG. 15 is a block diagram illustrating a data flow for processing intracellular activity with nanofid apparatus;

FIG. 16 is a block diagram illustrating an exemplary Fiber Bragg Grating (FBG) system;

FIG. 17 is a block diagram illustrating a functional layering for an exemplary nanofid apparatus;

FIG. 18 is a block diagram illustrating a functional layering for an exemplary nanofid apparatus;

FIGS. 19A and 19B are a flow diagram illustrating a method for a method for determining intracellular activity with nanofid apparatus;

FIG. 20 is a block diagram illustrating a nanofid injection system; and

FIG. 21 is a block diagram illustrating an exemplary nanofid injection system injection process.

### DETAILED DESCRIPTION OF PREFERRED EMBODIMENTS

### Exemplary Nanofid Apparatus

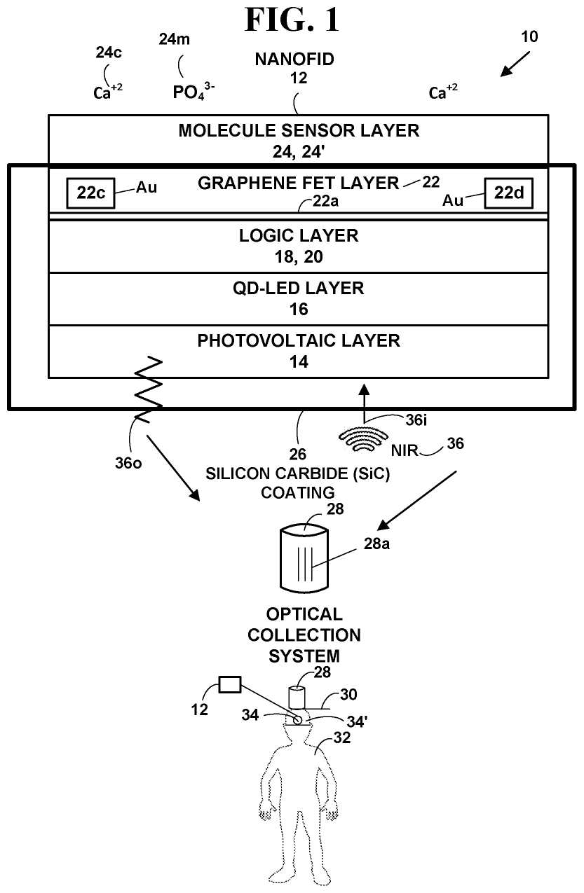

FIG. 1 is a block diagram 10 illustrating an exemplary nanofid apparatus 12.

The nanofid apparatus 12 includes, but is not limited to, a photovoltaic panel (PV) layer 14, a quantum dots (QD) light emitting diodes (LEDs) layer 16, a logic layer 18, a graphene Field Electric Transistor (FET) layer 22, a molecule sensor layer 24, a silicon carbide (SiC) coating 26. However, the present invention is not limited to such an embodiment and more, fewer and/or other layers can be used to practice the invention.

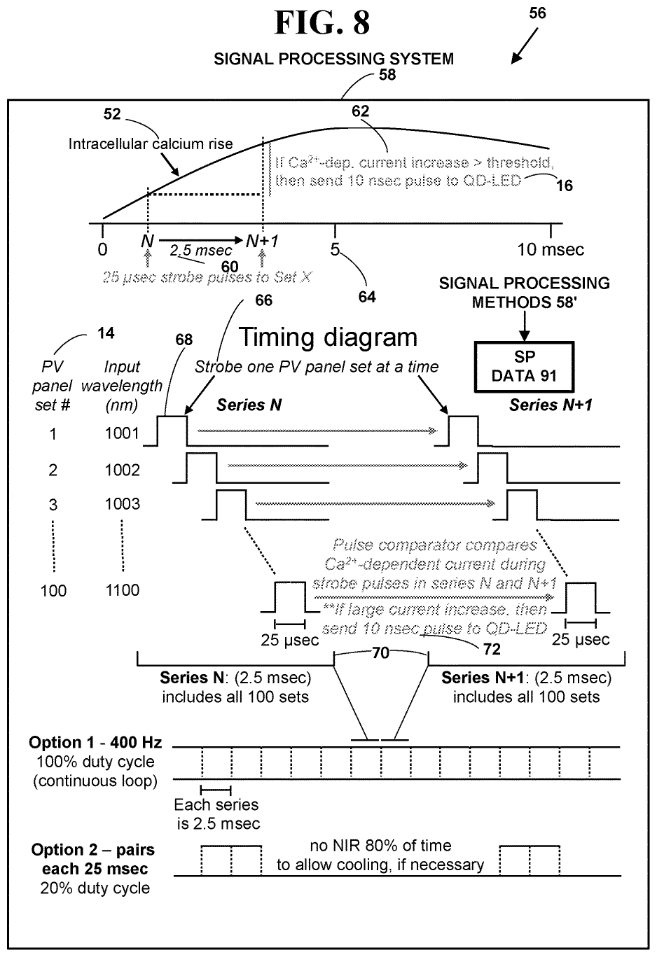

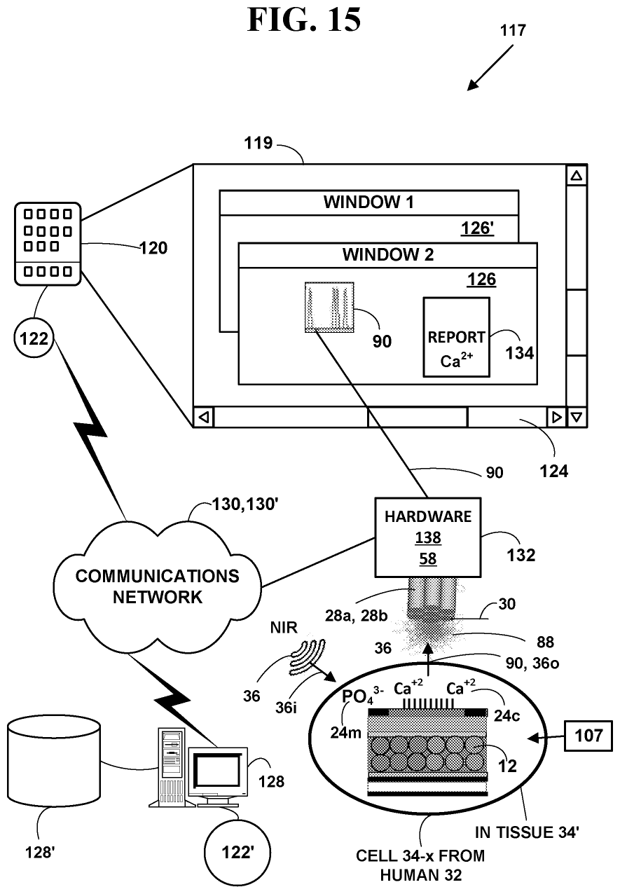

The nanofid apparatus 12 includes a nanofid system 117, including, but not limited to a plurality of nanofid apparatus 12, a nanofid external NIR light source 36 (FIG. 1), an external nanofid optical collection system 28, an external nanofid signal processing system 58 (FIG. 8) with one or more signal processing methods 58′, an external nanofid information data processing and display system 119 (FIG. 15). However, the present invention is not limited to such an embodiment and more, fewer and/or other system component can be used to practice the invention.

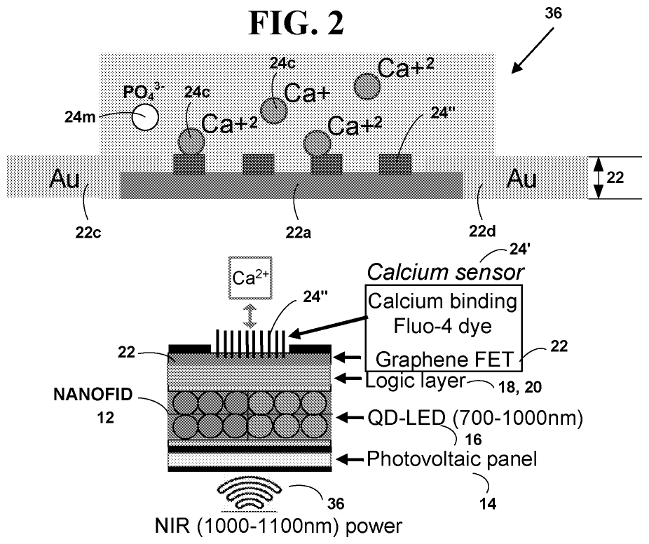

The nanofid 12 is inserted and used to detect and measure calcium molecules concentrations 24c (e.g., Ca²⁺, etc.) (FIG. 2), calcium ions, and/other intracellular molecules 24m (e.g., PO₄³⁻, etc.) (FIGS. 1 and 2) in individual cells 34 in a human being 32. In other embodiments, the nanofid apparatus 12 is used on other animals (e.g., rats, mice, monkeys, etc.) However, the present invention is not limited to such an embodiment and more, fewer and/or other layers can be used to practice the invention.

The intracellular molecules 24m including, PO₄³⁻, are sensed to evaluate kidney functions, disorders of the central nervous system, etc. However, the present invention is not limited to such an embodiment and more, fewer and/or other intracelluar molecules can be used to practice the invention.

In addition, the nanofid 12 apparatus is not limited to the layering and/or ordering of the layering shown and other layering and ordering of the layering can be used to practice the invention.

For example, in one specific embodiment, the logic layer 18 is placed below the PV layer 14. However, the present invention is not limited to such an embodiment, and other layering orders can be used to practice the invention.

A plurality of nanofid apparatus 12 are inserted directly into the plurality of pre-determined individual cell types 34 in the pre-determined biological tissue to monitor concentrations of pre-determined intracellular molecules 24c, 24m (e.g., Ca²⁺, PO₄³⁻, etc.) without causing any harm, toxicity or death to the plurality of pre-determined individual cell types 34 in the pre-determined biological tissue or the pre-determined biological tissue.

The photovoltaic (PV) panel layer 14, provides power to the nanofid apparatus 12 and includes but is not limited to, a photoelectric panel powered by near infrared (NIR) light 15 with a wavelength in a range of about 1000-1100 nanometers (10⁻⁹ meters (m)) from optical fibers 28a that activate quantum dots (QD) in the photovoltaic (PV) panel 14. The PV panels 14 can be linked in series to increase voltage. However, the present invention is not limited to such an embodiment and other embodiments can be used to practice the invention.

The optical fibers 28a, include, but are not limited to, for example, a seven-fiber optical fiber array with 1.5 millimeter (mm) diameter optical fibers include a maximum area (of about 19 mm²) of light scatter from each nanofid apparatus 12. However, the present invention is not limited to such an embodiment and other embodiments can be used to practice the invention.

In another embodiments, a continuous array 28b of a plurality of 1.5 mm optical fibers 28a is used for optical collection However, the present invention is not limited to such an embodiment and other embodiments can be used to practice the invention.

In an electrical circuit, connecting components in “series” means they are connected end-to-end, forming a single path for current flow, where a current passes through each component.

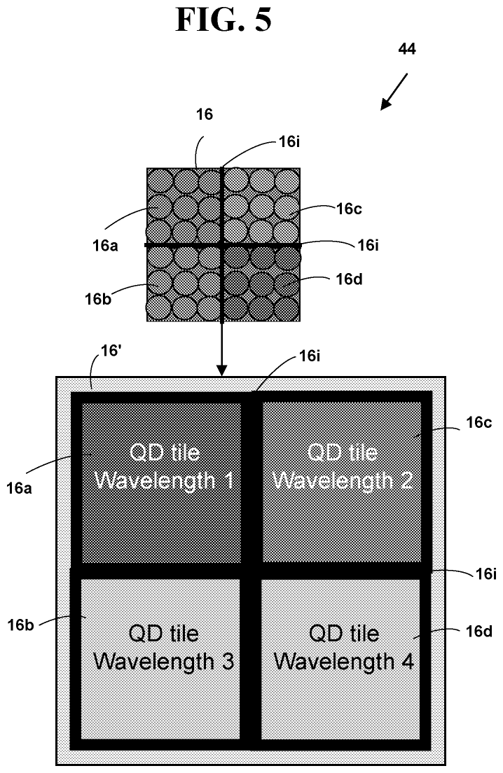

The quantum dots (QD) light emitting diodes (LEDs) layer 16, includes, but are not limited to, a plurality of different sized QD-LEDs separated by a photoresist insulator 16i. FIG. 5 illustrates four different sized QD-LEDs on four tiles 16a, 16b, 16c, 16d separated by a photoresist insulator 16i. In one embodiment, the QD-LED layer 16 in the QD-LED of each nanofids 12 is divided into four separate tiles 16-1, 16-2, 16-x, etc. separated by the photoresist insulator 16i. Only one size of QD is active in each tile. Different combinations of four QD sizes from 30 available QD sizes (10 nm bandwidth bins) emit about thirty different NIR wavelengths of about 700-1000 nm. However, the present invention is not limited to such embodiments and other embodiments including more, fewer and/or other types and numbers of QD-LEDs may be used to practice the invention.

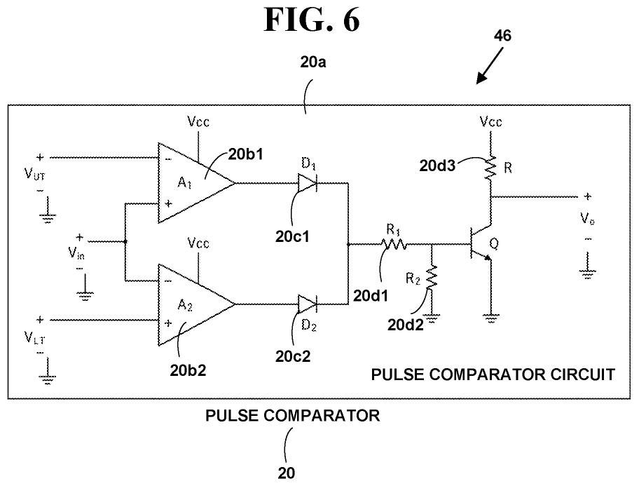

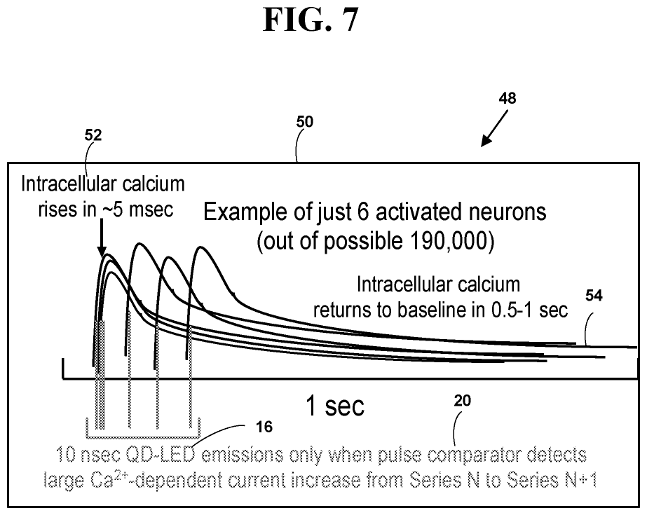

The logic layer 18 includes, but is not limited to, a pulse comparator 20 (FIG. 6 and FIG. 7). In this embodiment, a time course for output of near infrared (NIR) light emission from an activated nanofid 12 is reduced to a single ten nanosecond (nsec) (10⁻⁹) pulse. In this embodiment, the pulse comparator 20 receives and compares electrical current from a first twenty-five μsec pulse to a second twenty-five μsec pulse with strobed input coding. However, other embodiments with other comparisons can be used to practice the invention. If the electrical current increases and passes pre-determined current threshold, then the pulse comparator 20 activates the QD-LEDs 16 to emit NIR light for ten nsec. This reduces co-emissions from multiple activated nanofids 12 to a near zero value.

Voltage is measured in volts (V) and does not flow, whereas current is measured in amperes (A) and flows through a circuit (e.g., pulse comparator circuit 20a, etc.).

In one embodiment, different NIR input wavelengths 36 strobe and activate different sets of nanofid apparatus 12. Thin layer optical filters (e.g., from ALLUXA, etc.) are applied to the PV layer 14 so that only one wavelength (i.e., 1 nm bandwidth, etc.) can activate a desired set of nanofid apparatus 12 to provide strobed input coding.

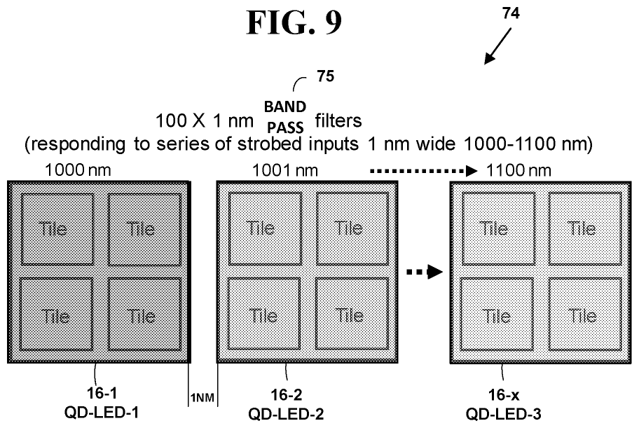

In the current embodiment, strobed input coding includes using 100×1-nm bandpass filters for strobed input coding to strobe and activate 100 different sets of nanofid apparatus 12 using 100 different wavelengths of about 1000-1100 nm (FIG. 9).

A bandpass filter is a type of filter that allows signals within a specific frequency range to pass through while blocking those outside of that frequency range. Essentially, it acts like a gate, letting only certain frequencies in.

The graphene Field Electric Transistor (FET) layer 22 includes a graphene layer 22a and gold (Au) electrodes 22c, 22d.

Graphene is a carbon allotrope consisting of a single layer of atoms arranged in honeycomb planar nanostructure. In one embodiment, graphene sheets are used. Graphene nanotubes can also be used. Graphene nanotubes are cylindrical structures formed by rolling up sheets of graphene. However, the present invention is not limited to such an embodiment and other embodiments may be used to practice the invention.

A Field Electric Transistor (FET), also known as a biosensor field-effect transistor (Bio-FET or BioFET) or field-effect biosensor (FEB), includes a field-effect transistor including a metal-oxide-semiconductor-field-effect transistor (MOSFET) structure that is gated by changes in surface potential induced by the binding of charged intracellular (i.e., within a cell 34, etc.) biomolecules to the graphene layer 22a.

When charged intracellular molecules 24m (e.g., PO₄³⁻, etc.) and other such as biomolecules (e.g., Ca²⁺ 24c, etc.) bind to a FET gate, which is usually a dielectric material, they change a charge distribution of the underlying semiconductor material resulting in a change in conductance of electrical current of an FET channel. A Bio-FET consists of two main components: (1) a biological recognition element (e.g., Fluo4, etc.) and (2) an electrical field-effect transistor (FET). However, the present invention is not limited to such an embodiment and other embodiments may be used to practice the invention.

In one embodiment, the molecule sensor 24 includes a general intracellular molecule sensor 24. The generalized intracellular molecule sensor 24 is used to sense different charged molecules, including, but not limited to, inorganic molecules, organic molecules, protein domains, immune molecules, antibody molecules, and/or other types of charged molecules, etc., that bind different intracellular molecules 24m (e.g., PO₄³⁻, etc.) that alter a charge on the graphene surface to alter electrical current through the graphene substrate. However, the present invention is not limited to such an embodiment and other embodiments may be used to practice the invention.

In one embodiment, the molecule sensor 24, includes, but is not limited to, a calcium sensor 24′ measuring increases in intracellular calcium concentrations 24c (e.g. calcium ion 2+, Ca²⁺, etc.) However, the present invention is not limited to such an embodiment and other embodiments can be used to practice the invention.

An increase in intracellular calcium concentrations 24c can occur through either an influx of calcium from the extracellular space or a release of calcium from intracellular stores, primarily from the endoplasmic or sarcoplasmic reticulum in the human body, including the brain, following electrophysiological activation of cells.

An increase in intracellular calcium can occur in the human body, including, but not limited to: (1) ATP Depletion: During ischemia (lack of oxygen), ATP levels fall, disrupting ion pumps and leading to calcium influx; (2) Sodium-Calcium Exchanger: A Na+/Ca²⁺ exchanger is a protein that exchanges sodium ions (Na+) for calcium ions (Ca²⁺) across the cell membrane. When intracellular sodium levels increase, the exchanger can facilitate calcium influx; (3) Hormones and Transmitters: Hormones and neurotransmitters can trigger intracellular calcium release or influx by activating specific receptors on the cell surface; (4) Cellular Stress: Various stressors, including hypoxia, oxidative stress, and inflammation, can contribute to increased intracellular calcium levels.

The calcium censor 24, includes, but is not limited to, calcium-binding molecule on the graphene FET layer 22. Increasing calcium inside an activated neuron in the human brain and/or other cell in the human body increases calcium bound to calcium-binding molecule on the surface of graphene FET 22. Each bound calcium ion Ca²⁺ brings 2+ charges to the surface of graphene to alter electrical current through the graphene substrate. Two chemical element gold (Au) blocks 22b, 22c are connected to a graphene sheet 22a in the graphene FET 22. The calcium binding molecule includes, for example, but is not limited to, a Fluo-4 dye. However, Fluo-4 is just one of hundreds of other different calcium binding molecules that can be used for the calcium binding molecule.

Fluo-4 is a cell-permeable Ca²⁺ indicator that is metabolized by intracellular esterase, leading to a bright green fluorescent signal upon Ca²⁺ binding (e.g., excitation/emission λ at 494/506 nm). Fluo-4 is used for visualization and measurement of intracellular Ca²⁺ during cell activation, particularly during neural activation. It selectively binds calcium with binding and release (Kon/Koff) properties that are well-suited for detecting alterations of intracellular calcium during normal neural activity.

Calcium-binding molecules are a diverse group of molecules that selectively bind calcium. They can be inorganic or organic molecules (e.g. Fluo4 or EGTA, etc.) or protein domains (e.g. calmodulin, etc.). However, the present invention is not limited to such embodiments and other embodiments can be used to practice the invention.

A protein domain is a distinct, compact, three-dimensional functional and/or structural unit within a protein that folds independently of a rest of the polypeptide chain.

Ethyleneglycol bis(2-aminoethyl ether)-N,N,N′,N′-tetraacetic acid (EGTA) is a calcium-specific chelating agent. It binds to calcium ions with a high affinity, effectively removing them from solution. This makes EGTA useful in various applications, including cell biology research, protein purification and other areas.

Calmodulin is a ubiquitous, calcium-binding protein that acts as a major calcium sensor in eukaryotic cells, regulating numerous cellular processes. It acts as an intermediary protein, sensing calcium levels and transmitting signals to other proteins, enzymes, and ion channels.

In one embodiment, other different molecules 24m (e.g., inorganic and organic molecules, protein domains and/or immune molecules, antibodies etc.) are attached to the graphene FET 22 to bind other different intracellular molecules 24m that alter the charge on the graphene surface to alter electrical current through the graphene substrate. However, the present invention is not limited to such embodiments and other embodiments can be used to practice the invention.

The nanofids 12 are sealed with a hermetic sealing including, but not limited to, a Silicon Carbide (SiC) coating 26. Silicon Carbide (SiC) 26 also known as carborundum is a hard chemical compound containing silicon and carbon. Silicon carbide exists in about 250 different crystalline forms.

All sides of the nanofids 12 are sealed with the SiC coating 26 except the calcium-binding 24′ surfaces. The SiC coating 26 is also applied to completely cover the gold (Au) electrodes 22c, 22d of FET 22, during a wafer manufacturing process. The SiC coating 26 also acts as an electrical insulator. However, the present invention is not limited to such an embodiment and other embodiments may be used to practice the invention.

A wafer manufacturing process for nanofids 12 involve several steps to transform raw silicon into the thin, polished discs used in semiconductor fabrication. These steps typically include ingot growth, slicing, lapping, etching, polishing, and cleaning. The final wafer is then ready for the processes of circuit design and fabrication for the nanofids 12.

The external optical collection system 28, includes plural optical collection fibers 28a in an optical fiber array 28b on an external surface 30 of a human body 32 (e.g., cortical surface of a human brain, etc.)

In one embodiment, the optical fiber arrays 28b, include, but are not limited to 1.5 mm diameter optical fibers placed on the external surface 30 of the human body 32 (e.g., on the cortical surface (not in the cortex) of a human brain, etc.) to collect NIR signals 36 from activated cells (e.g., activated neuron brain cells 34, etc.) below the optical fiber arrays 28a, which avoids cortical tissue damage in the human brain. The optical collection system 28 further includes a new beam splitter added to allow 1000-1100 nm input 36i power to the nanofids 12 and 700-1000 nm output 360 from nanofids 12.

FIG. 2. is a block 36 diagram illustrating an exemplary nanofid apparatus 12.

In FIG. 2, individual calcium sensors 24″ are illustrated in a calcium sensor layer 24′ in the

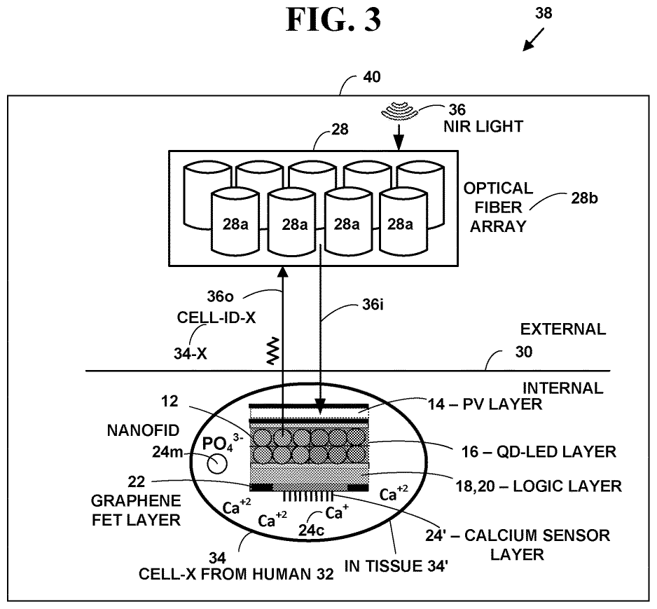

FIG. 3 is a block diagram 38 illustrating an exemplary nanofid apparatus system 40.

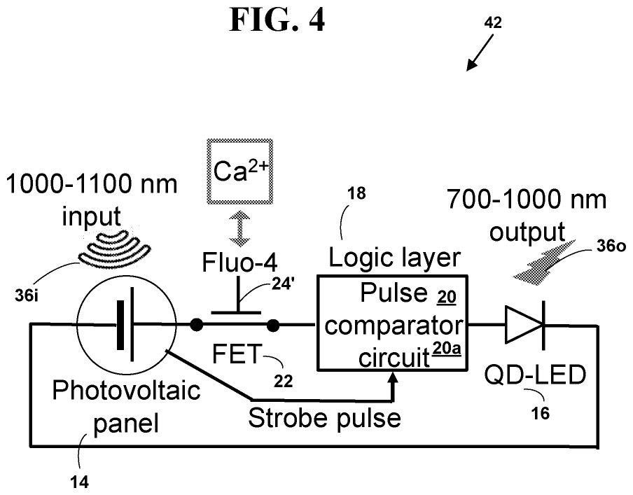

FIG. 4 is a block diagram 42 illustrating additional details of the exemplary nanofid apparatus system 40.

FIG. 5 is a block diagram 44 illustrating additional details of a Quantum Dot (QD) Light Emitting Diode (LED) 16.

FIG. 6 is a block diagram 46 illustrating additional details of a pulse comparator 20 of the exemplary nanofid apparatus 12.

IN FIG. 6, the pulse comparator 20 includes a pulse comparator circuit 20a with a plurality of amplifiers 20b1, 20b2, diodes 20c1, 20c2, and resistors 20d1, 20d2, 20d3.

FIG. 7 is a block diagram 48 illustrating a typical intracellular time course in activated cells (e.g. neurons) 50 and the function of the pulse comparator 20 to reduce the time for output from the exemplary nanofid apparatus 12.

Whatever the cause, when intracellular calcium levels rise 52 in about 5 msec in cells 34, (e.g., neurons, in a brain cortex of animals/humans, etc.). Intracellular calcium returns to a baseline level 54 in 0.5 to 1 second.

When intracellular calcium levels and/or other biological substance of interest reach or exceed a desired threshold value, the nanofid apparatus 12 detects and responds to such changes in threshold values.

FIG. 8 is a block diagram 56 illustrating a signal processing system 58 including pulse comparator timing diagram of the exemplary nanofid apparatus 12.

FIG. 8 includes an exemplary signal processing methods 58′ and signal processing data 91 produced by exemplary signal processing methods 58′.

It has been determined experimentally that 2.5 msec 60 is optimal for assessing Ca²⁺ dependent current change 62 in most cells 34. The 2.5 msec time 60 captures 50% of possible current increase 62 during a 5 msec 64 intracellular calcium rise.

The PV layer 14 is strobed 66 in a cell 34 with a NIR light pulse that produces a voltage pulse 68. The 2.5 msec 62 time always allows at least two voltage pulses 70 (e.g., N, N+1) to be compared during a 5 msec rise.

During the 1 sec time course for increased intracellular calcium in the average active neuron, up to 10% (190,000) of all 1.9 million neurons can be active under a 7-fiber optical fiber array 28a. All 1.9 million neurons can be activated within even a small bin of 25 msec. The problem is that no more than 2-3 co-emissions can be distinguished from the nanofids apparatus 12.

One solution to this problem is to reduce nanofids apparatus 12 emission to only ten nsec 72. Even if all 190,000 neurons are co-activated within 25 msec, then average spread is 132-1320 nsec. The ten nsec 72 emission ensures very few overlapping nanofid apparatus 12 emissions.

The ten nsec 72 QD-LED layer 16 emissions occur only when the pulse comparator 20 detects large Ca²⁺-dependent current increase 52 between two light pulses 70 from Series N to Series N+1. That is, if the Ca²⁺-dependent current increase 52 during the second light pulse is greater than the first light pulse by a pre-determined threshold value, then a ten nsec 72 pulse is sent to the QD-LED layer 16 of the nanofid apparatus 12 to activate sensing of pre-determined molecule of interest 24c (e.g., Ca²⁺, etc.) 24m in an individual cell 34 including the nanofid apparatus 12.

Table 1 illustrates an exemplary signal processing method 58′. However, the present invention is not limited to the steps illustrated in Table 1, and more, fewer and other steps in other orders and other signal processing methods 58′ and other embodiments can be used to practice the invention.

TABLE 1
1. A plurality of PV layers 14 in a plurality of nanofid apparatus 12 in a plurality of
individual cells 34 in a pre-determined tissue 34′ is strobed 66 by external NIR light
source 36 with a NIR light pulse 36i using a 2.5 msec time interval to provide power to
the plurality of nanofid apparatus 12 in the plurality of individual cells 34 in the pre-
determined tissue 34′. The 2.5 msec 62 strobe time always allows at least two voltage
pulses 70 (e.g., N, N + 1) created by the graphene Field Electric Transistor (FET) layer
22 and the molecule sensing layer 24, 24′ to be compared during a 5 msec rise in
concentrations in the pre-determined molecule of interest 24c (e.g., Ca²⁺, etc.) 24m.
2. After strobing the plurality of PV layer 14 activates the logic layer 18, 20, the
graphene Field Electric Transistor (FET) layer 22 and the molecule sensing layer 24,
24 in the plurality of nanofid apparatus 12 in the plurality of individual cells 34.
3. The graphene Field Electric Transistor (FET) layer 22 and the molecule sensing
layer 24, 24′ sense in the plurality of nanofid apparatus 12 in the plurality of different
individual cells 34 for in changes concentrations in the pre-determined molecule of
interest 24c, 24m and generate a plurality of voltage pulses to a plurality of logic layers
18, 20 in the plurality of nanofid apparatus 12 when the changes concentrations in the
pre-determined molecule of interest 24c, 24m are detected.
4. If a pulse comparator 20 in a logic layer 20 in nanofid apparatus 12 detects a current
increase 52 between the two strobed 66 pulses that exceeds a pre-determined threshold
value, then strobed input coding is generated with ten nsec light pulse using 100 × 1-
nm bandpass filters to strobe and activate 100 different sets of nanofids 12 in 100
different cells 34 using 100 different light pluse wavelengths of about 1000-1100 nm.
The strobed input coding is sent to a desired QD-LED tile 16a-16d in QD-LED layer
16 of the plurality of nanofid apparatus 12 to activate the desired QD-LED tile 16a-16d
in the plurality of nanofid apparatus 12. The strobed light pulse is sent to the desired
QD-LED tile 16a-16d in QD-LED layer 16 using a bandpass filter to select the desired
QD-LED tile 16a-16d.
5. The desired QD-LED tile 16a-16d in QD-LED layer 16 when strobed with the light
pulse emits one of about thirty different NIR light output 30o wavelengths of about
700-1000 nm. Each QD-LED tile 16a-16d emits a different NIR output 30o
wavelength. A different QD-LED tile 16a-16d in the QD-LED layer 16 is selected by
the pulse comparator 20 for each time the QD-LED layer 16 is activated in the plurality
of nanofid apparatus 12 for the plurality of individual cells 34.
6. The plurality of a nanofid apparatus 12 in the plurality of individual cells 34 emit
light scatter 88 with a unique NIR identifier 90 for a cell-X 34-x for detection on a
single optical fiber 92, 28a in the optical fiber array 28a in the nanofid optical
collection system 28.

FIG. 9 is a block diagram 74 illustrating additional details of an exemplary nanofid apparatus strobe coding method 75.

FIG. 9 illustrates a strobe coding method 75 in which 100×1-nm bandpass filters are used strobe/activate 100 different sets of nanofids 12 via the QD-LED layers 16 using 100 different wavelengths from 1000-1100 nm.

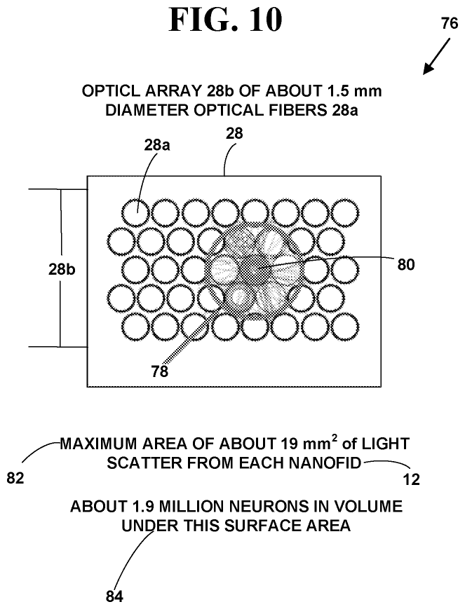

FIG. 10 is a block diagram illustrating light coupling into a nanofid optical collection system 28.

One problem with the nanofid optical collection systems 28 is that it is necessary to distinguish about 1.9 million unique nanofids 12 capable of being detected under one 7-fiber optical fiber array 78.

If the QDs in the QD-LED layer 16 are separated into 10 nm wavelength bins, this allows 40 unique QD-LED size/wavelengths in an NIR range 700-1100 nm. This produces 91,390 unique QD-LED combinations, but this is not enough because there 1.9 million unique neurons under a seven-fiber optical fiber array 78. A seven-fiber optical fiber array 78 with seven 1.5 mm diameter 80 optical fibers includes a maximum area of about 19 mm² 82 of light scatter from each nanofids apparatus 12. This results in about 1.9 million neurons in volume 84.

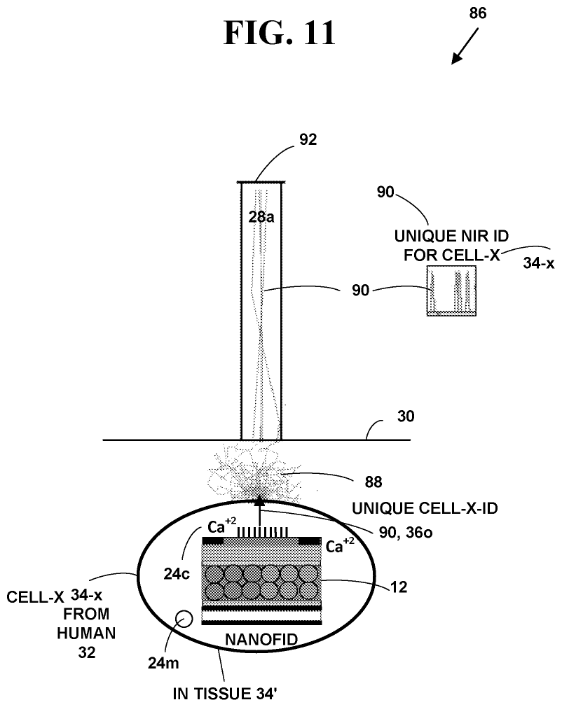

FIG. 11 is a block diagram 86 illustrating additional details of an exemplary nanofid apparatus 12 data flow.

In FIG. 11, a nanofid apparatus 12 emits light scatter 88 with a unique NIR identifier 90 for a cell-X 34-x for detection by a single fiber 92, 28a in the optical fiber array 28a.

In one embodiment, it has been determined experimentally that NIR 36 collection from 1 μm nanofid apparatus 12 to the external 30 optical fiber array 28a on a cortical surface of a human brain includes a seven-fiber optical array 78 as an optimum size because very little light from the nanofids apparatus 12, travels beyond an external seven-fiber optical fiber array 78. However, the present invention is not limited to such an embodiment and other embodiments with other sized external optical fiber array 28a may be used to practice the invention.

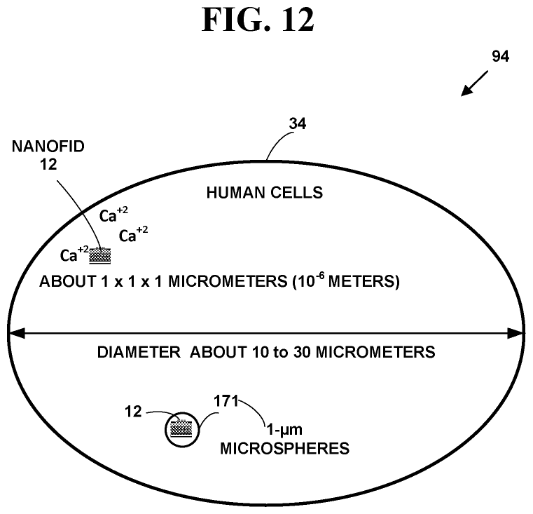

FIG. 12 is a block diagram 94 illustrating an exemplary nanofid apparatus 12 inserted in a human cell 34.

In FIG. 12, a human cell 34, on the average has a diameter of about 10 to 30 μm (10⁻⁶ meters). For the example application of measuring neural activity, pyramidal neurons in human cortex are approximately 17 μm. The nanofid apparatus 12 has a size of about 1 μm×1 μm×1 μm on each However, the present invention is not limited to such an embodiment and other embodiments with other sizes may be used to practice the invention.

### Exemplary Nanofid Apparatus Methods of Use

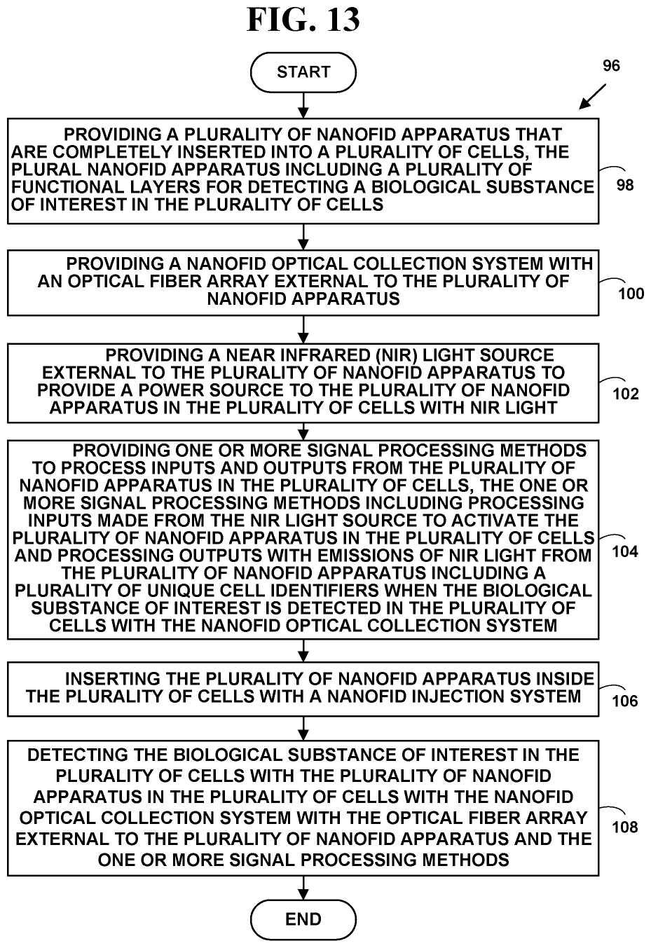

FIG. 13 is a flow diagram illustrating a Method 96 for determining intracellular activity with nanofid apparatus 12.

In FIG. 13, at Step 98 providing a plurality of nanofid apparatus that are completely inserted into a plurality of cells, the plural nanofid apparatus including a plurality of functional layers for detecting a biological substance of interest in the plurality of cells; at Step 100, providing a nanofid optical collection system with an optical fiber array external to the plurality of nanofid apparatus; at Step 102, providing a Near Infrared (NIR) light source external to the plurality of nanofid apparatus to provide a power source to the plurality of nanofid apparatus in the plurality of cells with NIR light; at Step 104, providing one or more signal processing methods to process inputs and outputs from the plurality of nanofid apparatus in the plurality of cells, the one or more signal processing methods including processing inputs made from the NIR light source to activate the plurality of nanofid apparatus in the plurality of cells and processing outputs with emissions of NIR light from the plurality of nanofid apparatus including a plurality of unique cell identifiers when the biological substance of interest is detected in the plurality of cells with the nanofid optical collection system; at Step 106, inserting the plurality of nanofid apparatus inside the plurality of cells with a nanofid apparatus injection system; at Step 108, detecting the biological substance of interest in the plurality of cells with the plurality of nanofid apparatus in the plurality of cells with the nanofid optical collection system with the optical fiber array external to the plurality of nanofid apparatus and the one or more signal processing methods.

Method 96 is illustrated with an exemplary embodiment. However, the present invention is not limited to such an embodiment and other embodiments can be used to practice the invention.

In such an exemplary embodiment in FIG. 13, at Step 98 providing a plurality of nanofid apparatus 12 that are completely inserted into a plurality of cells 34, the plural nanofid apparatus 12 including a plurality of functional layers 14-24 for detecting a biological substance 24c, 24m of interest in the plurality of cells 34.

At Step 100, providing a nanofid optical collection system 28 with an optical fiber array 28b external to the plurality of nanofid apparatus 12.

At Step 102, providing a Near Infrared (NIR) light source 36 external to the plurality of nanofid apparatus 12 to provide a power source to the plurality of nanofid apparatus 12 in the plurality of cells 34 with NIR light 36.

At Step 104, providing one or more signal processing methods to process inputs and outputs from the plurality of nanofid apparatus 12 in the plurality of cells, the one or more signal processing methods 58′ including processing inputs made from the NIR light source 36i to activate the plurality of nanofid apparatus 12 in the plurality of cells 34 and processing outputs with emissions of NIR light 360 from the plurality of nanofid apparatus 12 including a plurality of unique cell identifiers 34 when the biological substance of interest 24c, 24m is detected in the plurality of cells 34 with the nanofid optical collection system 28.

At Step 106, inserting the plurality of nanofid apparatus 12 inside the plurality of cells 34 with a nanofid injection system 107.

At Step 108, detecting the biological substance of interest 24c, 24m in the plurality of cells 34 with the plurality of nanofid apparatus 12 in the plurality of cells 34 with the nanofid optical collection system 28 with the optical fiber array 28b external to the plurality of nanofid apparatus 12 and the one or more signal processing methods 58′.

However, the present invention is not limited to this embodiment and more, fewer and/or other steps can be used to practice the invention.

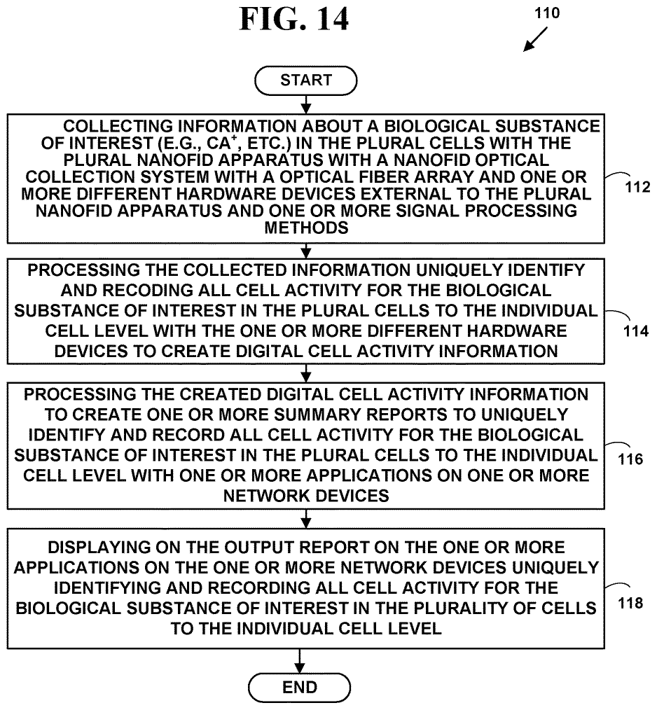

FIG. 14 is a flow diagram illustrating a Method 110 for intracellular activity with nanofid apparatus 12.

In FIG. 14 at Step 112, collecting information about the biological substance of interest in the plurality of cells with the plurality of nanofid apparatus with the nanofid optical collection system with the optical fiber array and one or more different hardware devices external to the plural nanofid apparatus and the one or more signal processing methods; at Step 114, processing the collected information uniquely identifying and recording all cell activity for the biological substance of interest in the plurality of cells to the individual cell level with a plurality of different hardware devices to create digital cell activity information about the plurality of cells; at Step 116, processing the collected digital cell activity information from the one or more different hardware devices uniquely identifying and recording all cell activity for the biological substance of interest in the plurality of cells to the individual cell level with one or more applications on one or more network devices, each with one or more processors to create an output report; and Step 118, displaying on the output report on the one or more applications on the one or more network devices uniquely identifying and recording all cell activity for the biological substance of interest in the plurality of cells to the individual cell level.

Method 110 is illustrated with an exemplary embodiment. However, the present invention is not limited to such an embodiment and other embodiments can be used to practice the invention.

In such an exemplary embodiment in FIG. 14 at Step 112, collecting information 360, 90 about the biological substance of interest 24c, 24m in the plurality of cells with the plurality of nanofid apparatus with the nanofid optical collection system 28 with the optical fiber array 28b and one or more different hardware devices 132, 133, 135, 138 external to the plural nanofid apparatus 12 and the one or more signal processing methods 58′.

At Step 114, processing the collected information 360, 90 uniquely identifying and recording all cell activity for the biological substance of interest 24c, 24m in the plurality of cells 34 to the individual cell 34 level with a plurality of different hardware devices 132, 133, 135, 138 to create digital cell 34 activity information about the plurality of cells 34.

At Step 116, processing the collected digital cell activity information 360, 90 from the one or more different hardware devices 132, 133, 135, 138 uniquely identifying and recording all cell er activity for the biological substance of interest 24c, 24m in the plurality of cells 34 to the individual cell 34 level with one or more applications 122, 122′ on one or more network devices 120, 128, each with one or more processors to create an output report 134.

At Step 118, displaying the output report 134 on the one or more applications 122, 122′ on the one or more network devices 120, 128 uniquely identifying and recording all cell 34 activity for the biological substance of interest 24c, 24m in the plurality of cells 34 to the individual cell 34 level.

However, the present invention is not limited to such an embodiment and other embodiments, with more, fewer and/or other hardware devices can be used to practice the invention.

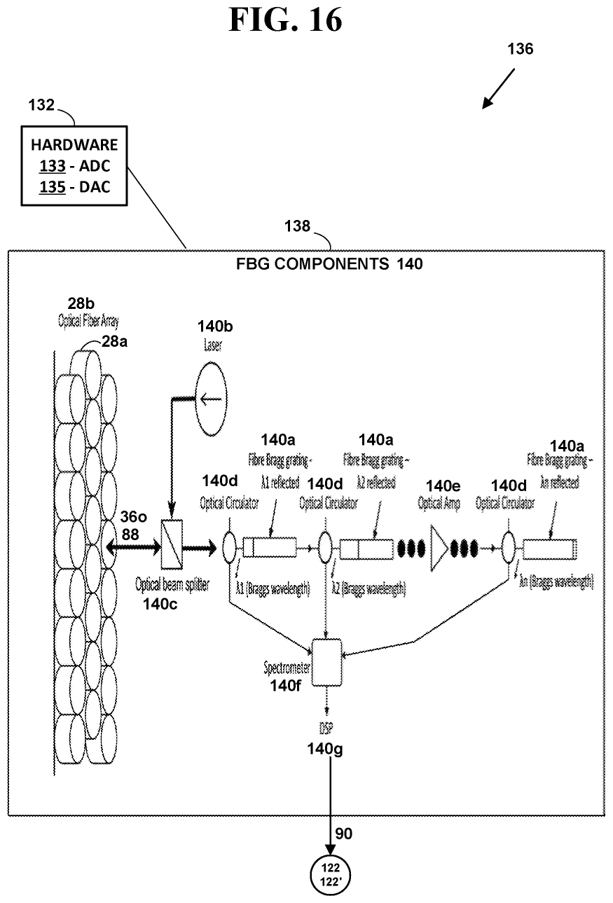

In one embodiment, at Step 112, about the biological substance of interest 24c, 24m in the individual cells 34 is collected using Fiber Bragg Grating (FBG) system 138 (FIG. 16). However, the present invention is not limited to such an embodiment and other embodiments can be used to practice the invention.

An FBG is a type of distributed Bragg reflector constructed in a short segment of optical fiber that reflects particular wavelengths of light and transmits all others. This is achieved by creating a periodic variation in the refractive index of the fiber core, which generates a wavelength-specific dielectric mirror. Therefore, a FBG is used as an inline optical filter to separate certain wavelengths, is used for sensing applications or it is used as wavelength-specific reflector. The Fibre Bragg Gratings (FBGs) 140a (FIG. 16) are used to sort the plurality nanofid apparatus 12 output NIR light data 360, 90 at a plurality of different wavelengths emitted by the plurality of nanofid apparatus 12 inserted into the plurality of individual cells 34 in the pre-determined biological tissue 34′ into distinct discrete information bins. One or more analog-to-digital convertors (ADC) 133 and digital-to-analog convertors (DAC) 135 are used to interface with the FBGs 140a to receive the output NIR light data 360 at the plurality of different wavelengths.

An Analog-to-Digital Converter (ADC) 133, includes a hardware device that transforms a continuous analog signal (e.g., voltage, current, etc.) into a digital representation of the continuous analog signal including discrete values (e.g., binary numbers like 101101). This process is necessary because most electronic systems and computing systems primarily operate with digital signals, making the conversion necessary for them to interact with real-world analog inputs.

A Digital-to-Analog Converter (DAC) 135 includes a hardware device that converts digital data (i.e., discrete numerical values) into a continuous analog signal. The digital data includes discrete values (e.g., binary numbers like 101101) and the continuous analog signal varies smoothly over time (e.g., voltage, current, etc.). A DAC 135 takes a digital input and produces a proportional analog output typically a voltage or current.

FIG. 15 is a block diagram 117 illustrating a nanofid information data processing and display system 119 for processing intracellular activity with nanofid apparatus 12.

The exemplary nanofid information data processing and system display system 119 includes, but is not limited to one or more target network devices (e.g., 120, etc. only one of which are illustrated for simplicity) each with one or more processors, memories and a non-transitory computer readable medium with an application 122 and a display component 124. The application 122 presents a graphical user interface (GUI) 126 on the display 122 component. The GUI 126 presents a multi-window 128, 128′ etc. (only two of which are illustrated) interface to a user.

In one embodiment of the invention, the application 122 is a software application. However, the present invention is not limited to this embodiment and the application 122 can be hardware, firmware, hardware and/or any combination thereof. However, the present invention is not limited these embodiments and other embodiments can be used to practice the invention.

In another embodiment, a portion of the application 122 is executing on the target network devices 120 and another portion of the application 122′ is executing on a server network device 128 with one or more processors. In another embodiment, both target network device 12 application 122 and server network device 128 application 122′ are stand-alone applications. However, the present invention is not limited these embodiments and other embodiments can be used to practice the invention.

In one embodiment, the application 122/122′ include non-cloud applications 122/122′. In another embodiment, application 122/122′ include cloud applications including Software as a Service (SaaS). However, the present invention is not limited these embodiments and other embodiments can be used to practice the invention.

In one embodiment the application 122/122′ produces an output report 134 including, but not limited to, uniquely identify and record all cortical neurons 34 in the human 32 brain (or brains of animals, etc.) with single neuron 34 resolution and/or records all desired cell 34 activity with single cell 34 resolution including measuring increases in intracellular calcium (e.g., Ca²⁺, etc.) concentrations. Single neuron 34 resolution is critical since because human behaviors and memories are encoded by sparse patterns of activated neurons called ‘ensembles’ that intermingle with other ensembles encoding different behaviors and memories. However, the present invention is not limited these embodiments and other embodiments can be used to practice the invention.

In another embodiment the application 122/122′ produces an output report 134 including, but not limited to, uniquely identify and record all cell 34 activity in a human 32 tissue/organ (or animal tissue/organ, etc.) with single cell 34 resolution and/or record all desired cell 34 activity for the biological substance of interest with single cell 34 resolution. However, the present invention is not limited these embodiments and other embodiments can be used to practice the invention.

In one embodiment, the output report 134 is created using an Artificial Intelligence (AI) application 122ai using Generative AI and/or Predictive AI in the applications 122,122′.

Generative AI (GenAI, or GAI) is a subset of AI that uses Generative AI models and Generative AI large language models (LLM) to produce new text, images, scans, videos, and/or other forms of data.

Predictive AI (PredAI or PAI) is also a subset of AI that uses Predictive AI models, and Predictive AI LLMs, machine learning and statistical analysis to forecast future events. Machine learning is a field of study in AI concerned with the development and study of statistical algorithms that can learn from data and generalize to unseen data, and thus perform tasks without explicit instructions.

In one embodiment, Generative AI model like CHATGPT including a medical data analytics LLMs is used in applications 122/122′ analyze data collected from sensing concentrations of the pre-determined intracellular molecules 24c, 24m in the plurality of individual cells 34 in the pre-determined biological tissue 34′ sensed by the plurality of nanofid apparatus 12 inserted into the plurality of individual cells 34 in the pre-determined biological tissue 12. Predictive AI is used provide visualizations in the form of graphs and charts in the output report 134 to help users understand results summarized in the output report 134. However, the present invention is not limited to such an embodiment and other embodiments, with and/or without AI can be used to practice the invention.

In one embodiment, the application 122 includes a standalone application 122 on a target network device 120 that is not used with a communications network 130. In another embodiment, the application 122 is in communications with a server network device 128 via the communications network 130. However, the present invention is not limited these embodiments and other embodiments can be used to practice the invention.

One or more server network devices 128 (only one of which is illustrated for simplicity) each with one or more processors and a non-transitory computer readable medium include one or more associated databases 128′. The server network device 128 is in communications with the one or more target devices 120 via a communications network 130 and/or a cloud communications network 130′.

An operating environment for the network devices includes a processing system with one or more high speed Central Processing Unit(s) (CPU), processors, one or more memories and/or other types of non-transitory computer readable mediums.

In accordance with the practices of persons skilled in the art of computer programming, the present invention is described below with reference to acts and symbolic representations of operations or instructions that are performed by the processing system, unless indicated otherwise. Such acts and operations or instructions are referred to as being “computer-executed,” “CPU-executed,” or “processor-executed.”

It will be appreciated that acts and symbolically represented operations or instructions include the manipulation of electrical information by the CPU or processor. An electrical system represents data bits which cause a resulting transformation or reduction of the electrical information or biological information, and the maintenance of data bits at memory locations in a memory system to thereby reconfigure or otherwise alter the CPU's or processor's operation, as well as other processing of information. The memory locations where data bits are maintained are physical locations that have particular electrical, magnetic, optical, or organic properties corresponding to the data bits.

The data bits may also be maintained on a non-transitory computer readable medium including magnetic disks, optical disks, organic memory, and any other volatile (e.g., Random Access Memory (RAM)) or non-volatile (e.g., Read-Only Memory (ROM), flash memory, etc.) mass storage system readable by the CPU. The non-transitory computer readable medium includes cooperating or interconnected computer readable medium, which exist exclusively on the processing system or can be distributed among multiple interconnected processing systems that may be local or remote to the processing system.

FIG. 16 is a block diagram 136 illustrating an exemplary Fiber Bragg Grating (FBG) system 138.

In FIG. 16, the FBG system 138, includes, but are not limited to, plural FBG components 140, including but not limited to, optical inputs 360, 90 from the optical fiber array 28b, FBG grates 140a, a laser component 140b, an optical beam splitter component 140c, plural optical circulators 140d, one or more optical amplifiers 140e, a spectrometer 140f and one or more digital signal processors (DSP) 140g. FBG system 138 receives light scatter 88 information from the optical fiber array 28b and collects the unique NIR identifiers 90 for single individual cells-X 34-x for further processing by applications 122,122′ and one or more other signal processing methods.

However, the present invention is not limited these embodiments and more, fewer, and other components can be included in the FBG system and other embodiments can be used to practice the invention.

### Exemplary Nanofid Apparatus Fabrication

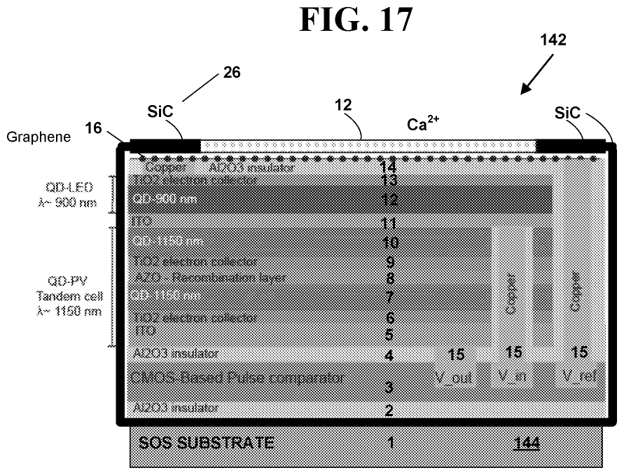

FIG. 17 is a block diagram 142 illustrating a functional layering for an exemplary nanofid apparatus 12. To manufacture an individual nanofid apparatus 12, the steps, include, but are not limited to the steps included in Table 2. However, the present invention is not limited to the steps illustrated in Table 2, and more, fewer and other steps in other orders and other embodiments can be used to practice the invention. In addition, nanofid apparatus 12 can be built including different layers added in different orderings and different sequences with different components.

TABLE 2

| | |
|---|---|
| 1. | Start with silicon on sapphire substrate (SOS) 144 |
| 2. | Ad an Aluminum Oxide Al₂O₃ insulator layer. |
| 3. | Add a CMOS-based pulse comparator 20a circuit layer. |
| 4. | Ad an Aluminum Oxide Al₂O₃ insulator layer. |
| 5. | Add an Indium Tin Oxide (ITO) layer. |
| 6. | Add a Titanium Oxide TiO₂ electron collection layer. |
| 7. | Add a QD 16 layer λ 1150 nm. |
| 8. | Add an AZO recombination layer. |
| 9. | Add a Titanium Oxide TiO₂ electron collection layer. |
| 10. | Add a QD 16 layer λ 1150 nm. |
| 11. | Add an Indium Tin Oxide (ITO) layer. |
| 12. | QD 16 layer λ 900 nm. |
| 13. | Add a Titanium Oxide TiO₂ electron collection layer. |
| 14. | Ad an Aluminum Oxide Al₂O₃ insulator layer with a copper |
| | connector. |
| 15. | Add an end of a V_out copper connector, V_in copper connector, |
| | V_ref copper connector to the CMOS-based pulse comparator 20a |
| | circuit. |
| 16. | Add a graphene layer 22. |
| 17. | Add a Calcium sensor layer 24. |
| 18. | The last layer will be Silicon Carbide (SiC) 26 covering the copper |
| | electrodes, |
| 19. | with a temporary mask (that is dissolved later) over the central |
| | graphene layer(s) 22. |

Layers 4-9 comprise a QD 16/PV 14 tandem cell with λ 1150 nm.
Layers 11-13 comprise a QD-LED 16 at λ 900 nm

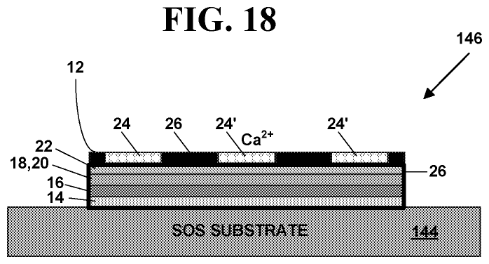

FIG. 18 is a block diagram 146 illustrating a functional layering for an exemplary nanofid apparatus 12. The post wafter manufacturing steps of nanofid apparatus 12, include, but are not limited to the steps included in Table 3. However, the present invention is not limited to the steps illustrated in Table 3, and more, fewer and other steps in other orders and other embodiments can be used to practice the invention.

TABLE 3

| | |
|---|---|
| 1. | Start with layers on SOS substrate 144 wafer. |
| 2. | Attach temporary transfer substrate to hold nanofid apparatus 12 |
| | during further steps. |
| 3. | Laser ablation of SOS substrate 144. Any remaining SOS is fine since |
| | it insulates but it should be insignificant thickness. |
| 4. | Use plasma laser to single devices on transfer substrate. |
| 5. | Apply thin layer optical filter (1 nm bandpass filter, from 1001-1100 |
| | nm wavelength). |
| 6. | Conformal coat all exposed surfaces with SiC 28 except calcium |
| | sensor 24c. |
| 7. | Release single nanofid apparatus 12 from transfer substrate. |

The nanofid apparatus 12, solution processing steps, include, but are not limited to the steps included in Table 4. However, the present invention is not limited to the steps illustrated in Table 4, and more, fewer and other steps and other embodiments can be used to practice the invention.

TABLE 4
1. Start with the singled nanofids 12 from post-wafer manufacturing steps.
2. Dissolve mask over graphene layer 22 with mask-specific solvent.
3. Wash solvent from nanofids 12 one time with water and remove solvent
traces with desalting column.
4. React Fluo4 or other calcium binding molecule (inorganic or organic) in
calcium sensor layer 24c with top graphene layer 22.
5. Wash solvent from devices 2 times with water and remove reactants and
solvent traces with desalting column.

Table 5. summarizes some of the characteristics of nanofid apparatus 12. However, the present invention is not limited to the steps illustrated in Table 5, and more, fewer and other characteristics and other embodiments can be used to practice the invention.

TABLE 5

| Characteristic | Nanofid Apparatus 12 |
|---|---|
| Resolution | Single Cell 34 |
| Scalability | Billions of Individual Cells 34 |
| Cell Access | Any Cell 34 in any Tissue |
| Size | 1 μm × 1 μm × 1 μm (10⁻⁶ meters) |
| Volume | 1 μm³ per cell 34 |
| Layering | Multi-layer with separate functional |
| | layers 14-26 |
| Cell to Detector | All Optical 28 |
| Information Transmission | |
| Signal Processing | NIR Band Pass Filtering with Buffering to |
| | external computer 120, 128 |
| Noise | Cell 34 movement and not Optical |
| | Processing |
| Power | NIR Light 36 |
| Invasiveness in Living Cells | Only during insertion |
| Long Term Living Cell and | Negligible to very low |
| Tissue Damage | |

### Exemplary Nanofid Apparatus Systems and Methods of Use

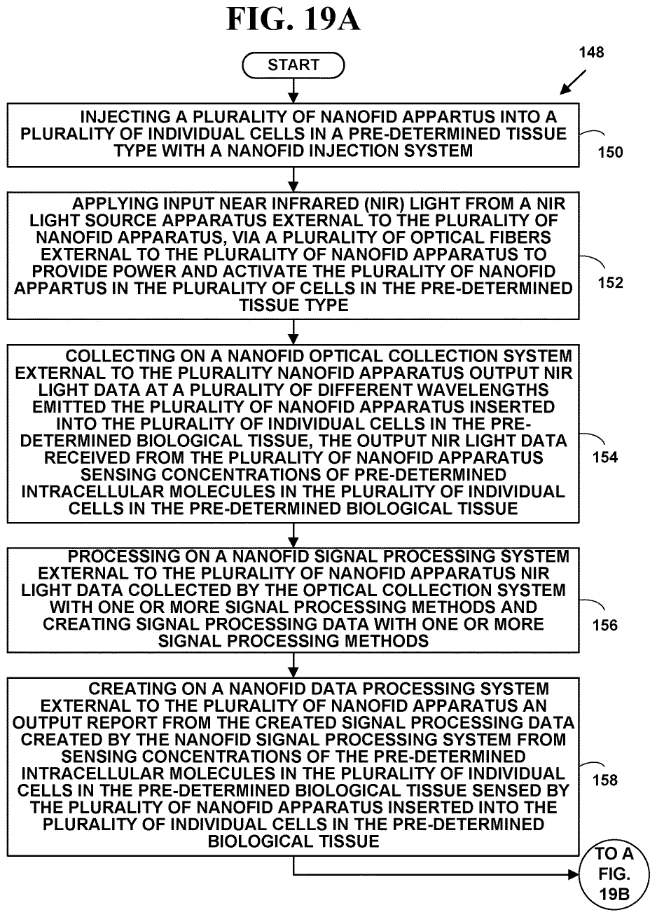

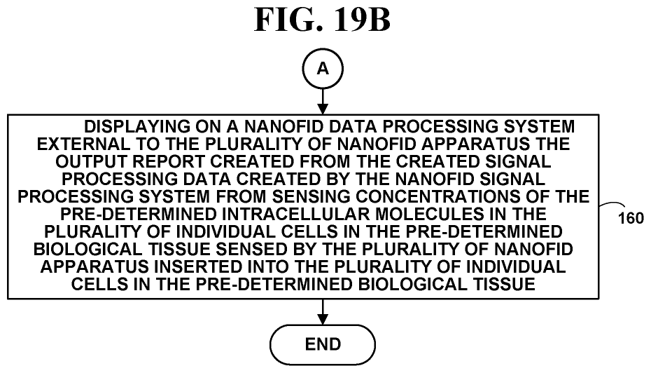

FIG. 19A is are a flow diagram illustrating a Method 148 for determining intracellular activity with nanofid apparatus.

In FIG. 19A at Step 150, injecting a plurality of nanofid apparatus into a plurality of individual cells in a pre-determined tissue type with a nanofid injection system; At Step 152, applying input Near InfraRed (NIR) light from a NIR light source apparatus external to the plurality of nanofid apparatus, via a plurality of optical fibers external to the plurality of nanofid apparatus to provide power and activate the plurality of nanofid apparatus in the plurality of cells in the pre-determined tissue type; At Step 154, collecting on a nanofid optical collection system external to the plurality nanofid apparatus output NIR light data at a plurality of different wavelengths emitted the plurality of nanofid apparatus inserted into the plurality of individual cells in the pre-determined biological tissue, the output NIR light data received from the plurality of nanofid apparatus sensing concentrations of pre-determined intracellular molecules in the plurality of individual cells in the pre-determined biological tissue; At Step 156 processing on a nanofid signal processing system external to the plurality of nanofid apparatus the output NIR light data collected by the optical collection system with one or more signal processing methods and creating signal processing data with one or more signal processing methods; at Step 158, creating on a nanofid data processing system external to the plurality of nanofid apparatus an output report from the created signal processing data created by the nanofid signal processing system from sensing concentrations of the pre-determined intracellular molecules in the plurality of individual cells in the pre-determined biological tissue sensed by the plurality of nanofid apparatus inserted into the plurality of individual cells in the pre-determined biological tissue; and at Step 160, displaying on the nanofid data processing system external to the plurality of nanofid apparatus the created output report created from the created signal processing data created by the nanofid signal processing system from sensing concentrations of the pre-determined intracellular molecules in the plurality of individual cells in the pre-determined biological tissue sensed by the plurality of nanofid apparatus inserted into the plurality of individual cells in the pre-determined biological tissue.

Method 146 is illustrated with an exemplary embodiment. However, the present invention is not limited to such an embodiment and other embodiments can be used to practice the invention.

In such an exemplary embodiment in FIG. 19 at Step 150, injecting a plurality of nanofid apparatus 12 into a plurality of individual cells 34 in a pre-determined tissue type 34′ with a nanofid injection system 107.

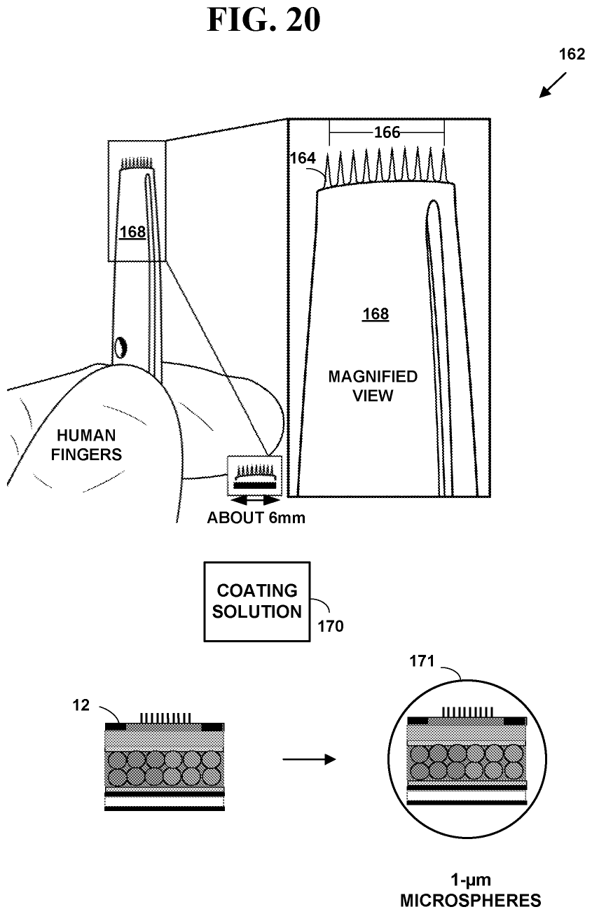

FIG. 20 is a block diagram 162 illustrating a nanofid injection system 107.

FIG. 20 illustrates plural microneedles 164 in a microneedle array 166 and microneedle insertion tool 168 and a coating solution 170 used to attach the nanofid apparatus 12 to the microneedles 164. In FIG. 20, the microneedle array 166 includes a width of about six millimeters. However, the present invention is not limited to such an embodiment and other embodiments, with more, fewer or components and sizes may be used to practice the invention.

In one embodiment, the nanofid injection system 107 includes plural nanofids 12 that are mixed into a 20% polyethylene glycol (PEG) 35K coated solution 170. Rigid arrays 166 of microneedles 164 in an injection tool 168 are coated with the nanofid 12 and PEG mixture 170 and the microneedles 164 are dried overnight. In one embodiment, the microneedles 164, include a length of about 25 to 1000 micrometers (μm), a Base width (i.e., a diameter) of about 50-300 μm and a tip radius less than about 10 μm.

In FIG. 20, a microneedle array 166 of ten microneedles 164 is illustrated. However, the present invention is not limited to a microneedle array 166 of this size and other microneedle arrays 166 of other size can be used to practice the invention. The microneedle array 166 with the plural nanofids 12 is then inserted into individual cells 34 (e.g., neuron cells, etc.) of the pre-determined tissue 34′ (e.g., cortical tissue 34′ of the human 32 brain) and/or other tissues 34′ sites in the human 32 body with the injection tool 168. The PEG solution is allowed to dissolve for about 15 minutes and to release the plural nanofids 12 into the individual cells 34 of the pre-determined tissue 34′. The microneedle array 166 in the injection tool 168 is then removed and the wound site is closed on the human 32 body.

In one embodiment, the plurality of nanofid apparatus 12 are enclosed in 1-μm (10⁻⁶ m) microspheres 171 before coating and injection. It has been determined experimentally, after injection, that the 1-μm microspheres 171 occupy only about 0.1% of a cell's soma of a typical about 17-μm diameter pyramidal neuron brain cell 34 (e.g., FIG. 12, etc.), and the 1-μm microspheres 171 including the plurality of nanofid apparatus 12 were observed experimentally in locations in the cytoplasm of the cells 34 and were not associated with any organelles. However, the present invention is not limited to such embodiment and other embodiments using other coating and injection methods and/or with and/or without the 1-μm microspheres can be used to practice the invention.

A soma (also called a cell body) is a central part of a neuron cell 34. A cytoplasm is a gel-like substance inside the cell 34 that fills the space between the cell membrane and the cell nucleus. The cytoplasm is where most of the cell's internal activity occurs. Cell organelles are specialized structures inside a cell 34 that perform specific jobs and include structures like mitochondria, ribosomes, endoplasmic reticulum, etc.

In one embodiment, the 1-μm microspheres 171 include biodegradable materials and/or inert materials. The biodegradable materials include, but are not limited to, polymers including Polylactic Acid (PLA) and/or Poly(lactic-co-glycolic acid (PLGA) 1-μm microspheres 171. PLA and PLGA are FDA-approved, biodegradable synthetic polymers widely used in medical applications. The inert materials include silica materials. The silica materials include, but are not limited to, silica microspheres are precision-engineered microparticles composed of synthetic amorphous silicon dioxide (SiO₂).

However, the present invention is not limited to such an embodiment and other nanofid injection system 107 components, injection microneedle sizes, injection microneedle array sizes, injection fluids, injection fluid concentrations, injection tools, and other injection microneedle sizes, injection microneedle array sizes, injection fluids, injection fluid concentrations, injection tools and other injection methods can be used to practice the invention.

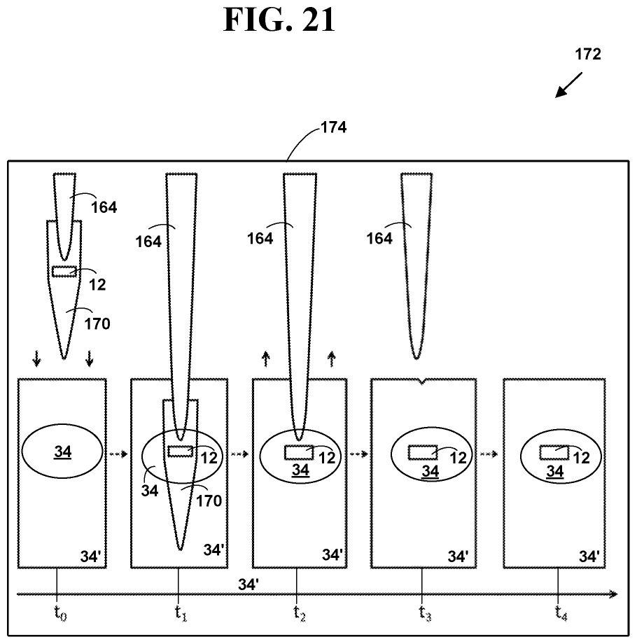

FIG. 21 is a block diagram 172 illustrating an exemplary nanofid injection system injection process 174.

In FIG. 21, an individual microneedle 164 coated with the coating solution 170 including the nanofid apparatus 12 is ready for insertion into a cell 34 of a pre-determined tissue 34′ at time to. At time t₁, the individual microneedle 164 coated with the coating solution 170 (e.g., PEG, etc.) including the nanofid apparatus 12 is inserted into the cell 34 of the pre-determined tissue 34′. At time t₂, individual microneedle 164 with the coating solution inserted into the cell of the pre-determined tissue 34′ is allowed to dissolve and to release the nanofid apparatus 12 into the individual cell 34 of the pre-determined tissue 34′. At time t₁, the individual microneedle 164 is removed from the individual cell 34 of the pre-determined tissue 34′. At time t₄, the nanofid apparatus 12 in the individual 34 of the pre-determined tissue 34′ is ready for activation by NIR light 36 and sensing of concentrations of pre-determined intracellular molecules 24m. FIG. 21 includes an expanded point of view and is not drawn to scale.

Returning to FIG. 19A at Step 152, applying input Near InfraRed (NIR) light 30i from a NIR light source apparatus 36 external to the plurality of nanofid apparatus 12, via a plurality of optical fibers 28 external to the plurality of nanofid apparatus 12 to provide power and activate the plurality of nanofid apparatus 12 in the plurality of cells 34 in the pre-determined tissue type 34′.

At Step 154, collecting on a nanofid optical collection system 28 external to the plurality nanofid apparatus 12 output NIR light data 360 at a plurality of different wavelengths emitted the plurality of nanofid apparatus 12 inserted into the plurality of individual cells 34 in the pre-determined biological tissue 34, the output NIR light data 360 received from the plurality of nanofid apparatus 12 sensing concentrations of pre-determined intracellular molecules 24m (e.g., Ca²⁺, PO₄³⁻, other inorganic molecules, organic molecules, protein domains, immune molecules, antibody molecules, etc.) in the plurality of individual cells 34 in the pre-determined biological tissue 34′.

At Step 156, processing on a nanofid signal processing system 58 external to the plurality of nanofid apparatus 12 the output NIR light data 360 collected by the nanofid optical collection system 28 with one or more signal processing methods 58′ and creating signal processing data 91 with one or more signal processing methods 58′.

At Step 158, creating on a nanofid data processing system 119 external to the plurality of nanofid apparatus 12 an output report 134 from the created signal processing data 91 created by the nanofid signal processing system 119 from sensing concentrations of the pre-determined intracellular molecules 24m (i.e., the biological substance of interest) in the plurality of individual cells 34 in the pre-determined biological tissue 34′ sensed by the plurality of nanofid apparatus 12 inserted into the plurality of individual cells 34 in the pre-determined biological tissue 34.

In FIG. 19B, at Step 160, displaying on the nanofid data processing system 119 external to the plurality of nanofid apparatus the created output report 134 created from the created signal processing data 91 created by the nanofid signal processing system 119 from sensing concentrations of the pre-determined intracellular molecules 24m in the plurality of individual cells 34 in the pre-determined biological tissue 34′ sensed by the plurality of nanofid apparatus 12 inserted into the plurality of individual cells 34 in the pre-determined biological tissue 34′.

The present invention was described in part, with respect to neurons and detecting desired levels of Cat in neurons in a brain of a human and/or animal. However, the present invention is not limited to such embodiments and other embodiments with other types of living cells and other biological substances of interest and detecting other types of ions and molecules can be used to practice the invention.

The apparatus and methods disclosed herein in intracellular monitoring device include plural multilayer nanofid apparatus 12 that fit completely inside plural living cells 34, and cause no significant impairment, to the plural cells 34 normal biological processes. The plural nanofid apparatus 12 monitor plural cells 34 for their level of a biological substance of interest 24c, 24m. If the biological substance of interest 24c, 24m, reaches or exceeds a threshold, the plural nanofid apparatus 12 in the plural living cells 34 transmits an electromagnetic signal (e.g., NIR light 360, etc., received by an optical collection system 28. Each nanofid apparatus 12 has its electromagnetic signal encoded with a unique frequency to uniquely identify it at a an individual cell level 34.

Detection of the frequency components, in the signals received by the optical collection system 28 with the signal processing methods 58′, permits identification of the source nanofid apparatus 12 in plural individual living cells 34 at the individual cell level 34.

It should be understood that the architecture, programs, processes, methods and systems described herein are not related or limited to any particular type of nanofid apparatus 12 components, layering, structures, chemical ions, molecules, compounds, signal processing methods, computer or network system (hardware or software), unless indicated otherwise. Various types of nanofid apparatus 12 components general purpose or specialized hardware and computer systems may be used with or perform operations in accordance with the teachings described herein.

In view of the wide variety of embodiments to which the principles of the present invention can be applied, it should be understood that the illustrated embodiments are exemplary only, and should not be taken as limiting the scope of the present invention. For example, the steps of the flow diagrams may be taken in sequences other than those described and more or fewer elements may be used in the block diagrams.

While various elements of the preferred embodiments have been described as being implemented in software, in other embodiments hardware or firmware implementations may alternatively be used, and vice-versa.

The claims should not be read as limited to the described order or elements unless stated to that effect. In addition, use of the term “means” in any claim is intended to invoke 35 U.S.C. § 112, paragraph 6, and any claim without the word “means” is not so intended. Therefore, all embodiments that come within the scope and spirit of the following claims and equivalents thereto are claimed as the invention.

We claim:

1. A nanofid apparatus, comprising in combination:
a photovoltaic panel (PV) layer, including a photoelectric panel with a plurality of different layers, powered by near infrared (NIR) light from a NIR light source apparatus external to the nanofid apparatus, configured for providing power including an electrical current to the nanofid apparatus;
a quantum dots (QD) light emitting diodes (LEDs) layer, including a plurality of QD-LEDs, configured for emitting NIR light at a plurality of different wavelengths when activated by a Field Electric Transistor (FET) layer and a molecule sensor layer;
a logic layer, including a pulse comparator, configured for comparing electrical current pulses generated from the PV layer with strobed by light using strobed input coding and activating one or more of the plurality of QD-LEDs when the electrical current increases and exceeds a pre-determined electrical current threshold;
the Field Electric Transistor (FET) layer, including an electrical biosensor field-effect transistor with a transistor gate and a graphene layer, configured for detecting changes in a surface potential of the graphene layer induced by binding of pre-determined intracellular molecules on the molecule sensor layer;
the molecule sensor layer, including a plurality of molecule sensors for sensing concentrations of the pre-determined intracellular molecules in a pre-determined biological tissue, configured for sensing changes in electrical charges of the concentrations of the pre-determined intracellular molecules detected on a surface of graphene layer that alters electrical current through the graphene layer sensing concentrations of the pre-determined intracellular molecules in the pre-determined biological tissue; and
a silicon carbide (SiC) coating, including an inert coating biocompatible for a plurality of different pre-determined types of biological tissue, configured for coating and protecting the photovoltaic panel (PV) layer, QD-LED layer, logic layer and FET layer of the nanofid apparatus within the pre-determined biological tissue,
wherein a plurality of the nanofid apparatus are inserted directly into a plurality of individual cells in the pre-determined biological tissue for sensing concentrations of the pre-determined intracellular molecules in the plurality of individual cells in the pre-determined biological tissue, without causing any harm, toxicity or death to the plurality of individual cells in the pre-determined biological tissue or the pre-determined biological tissue.

2. The nanofid apparatus of claim 1, wherein the PV layer includes: a photoelectric panel powered by the NIR light apparatus external to the nanofid apparatus with input wavelengths of about 1000-1100 nanometers (10⁻⁹ meters) via optical fibers that activate quantum dots (QD) in the photovoltaic (PV) panel to produce electrical power for the nanofid apparatus.

3. The nanofid apparatus of claim 1, wherein optical fibers include a seven-fiber optical fiber array with about 1.5 millimeter (mm) diameter optical fibers including a maximum area of about 19 mm² of light scatter from each nanofid apparatus and further including a continuous array of 1.5 mm optical fibers used for optical collection of the NIR light.

4. The nanofid apparatus of claim 1, wherein the QD-LED layer includes a plurality of different sized QD-LEDs on a plurality of tiles separated by a plurality of photoresist insulators in 10 nanometer (10⁻⁹ m) bandwidth bins, only one size of the QD-LEDs is active in each tile, different combinations of the plurality of different sized QD-LEDs sizes emit a plurality of different NIR output wavelengths in a range of about 700-1000 nanometers (10⁻⁹ meters) sensing concentrations of the pre-determined intracellular molecules in the pre-determined biological tissue.

5. The nanofid apparatus of claim 1, wherein the logic layer, including the pulse comparator compares electrical current from a first twenty-five microsecond (μsec) (10⁻⁶ seconds) pulse to a second twenty-five μsec pulse from pulse series-N to pulse series-N+1, with strobed input coding and if an electrical current increases and exceeds a pre-determined current threshold, then the pulse comparator activates a plurality of QD-LEDs to emit NIR light for ten nanosecond (10⁻⁹ seconds), reducing co-emissions from multiple activated nanofid apparatus inserted into a plurality of pre-determined cell types in the pre-determined biological tissue to a near zero value.

6. The nanofid apparatus of claim 5, wherein the strobed input coding includes using a plurality of bandpass filters to strobe and activate a plurality of different sets of nanofid apparatus inserted into a plurality of pre-determined cell types in the pre-determined biological tissue using a plurality of different wavelengths.

7. The nanofid apparatus of claim 6, wherein the plurality of bandpass filters includes a plurality of one nanometer (10⁻⁹ meter) bandpass filters to strobe and activate a plurality of different QD-LED layers within plurality of sets of nanofid apparatus using a plurality of different wavelengths of about 1000-1110 nanometers (10⁻⁹ meters).

8. The nanofid apparatus of claim 1, wherein the FET layer includes the biosensor field-effect transistor comprising a metal-oxide-semiconductor-field-effect transistor (MOSFET) structure with gold electrodes that is gated by changes in a surface potential induced by binding of the pre-determined intracellular molecules to the graphene layer.

9. The nanofid apparatus of claim 1, wherein the molecule sensor layer includes a plurality of molecule sensors for sensing a plurality of intracellular molecules including inorganic molecules, organic molecules, protein domains, immune molecules or antibody molecules.

10. The nanofid apparatus of claim 1, wherein the molecule sensor layer includes a plurality of calcium molecule sensors for sensing a plurality of intracellular charged calcium molecules.

11. The nanofid apparatus of claim 10, wherein the plurality of calcium molecule sensors sensing a plurality of intracellular charged calcium molecules produced by neuron cell activation in brain tissue of a human being or brain tissue of an animal.

12. The nanofid apparatus of claim 1, wherein the silicon carbide (SiC) coating includes coating all portions of the nanofid apparatus except the molecule sensor layer and wherein the SiC coating acting as an electrical insulator and is applied to the nanofid apparatus during a wafer manufacturing process.

13. The nanofid apparatus of claim 1, wherein a size of the nanofid apparatus includes a size of about one micrometer (10⁻⁶ meter) on each side to fit within a pre-determined individual cell types in the pre-determined biological tissue, the pre-determined individual cell types including a diameter of about ten to thirty micrometers.

14. The nanofid apparatus of claim 1, wherein a plurality of nanofid apparatus are inserted directly into the plurality of pre-determined individual cell types in the pre-determined biological tissue to monitor concentrations of pre-determined intracellular molecules without causing any harm, toxicity or death to the plurality of pre-determined individual cell types in the pre-determined biological tissue or to the pre-determined biological tissue.

15. The nanofid apparatus of claim 1, wherein a plurality of nanofid apparatus are enclosed in a plurality of one-μm (10⁻⁶ m) microspheres before insertion directly into the plurality of pre-determined individual cell types in the pre-determined biological tissue.

16. A nanofid apparatus system, comprising in combination:
a plurality of nanofid apparatus including:
a photovoltaic panel (PV) layer, including a photoelectric panel with a plurality of different layers, powered by near infrared (NIR) light from a NIR light source apparatus external to the plurality nanofid apparatus, configured for providing power including an electrical current to the plurality nanofid apparatus,
a quantum dots (QD) light emitting diodes (LEDs) layer, including a plurality of QD-LEDs, configured for emitting NIR light at a plurality of different wavelengths when activated by a Field Electric Transistor (FET) layer and a molecule sensor layer,
a logic layer, including a pulse comparator, configured for comparing electrical current pulses generated from the PV layer with strobed light using strobed input coding and activating one or more of the plurality of QD-LEDs when the electrical current increases and exceeds a pre-determined electrical current threshold,
the Field Electric Transistor (FET) layer, including an electrical biosensor field-effect transistor with a transistor gate and a graphene layer, configured for detecting changes in a surface potential of the graphene layer induced by binding of pre-determined intracellular molecules on the molecule sensor layer,
the molecule sensor layer, including a plurality of molecule sensors for sensing concentrations of the pre-determined intracellular molecules in a pre-determined biological tissue, configured for sensing changes in electrical charges of the concentrations of the pre-determined intracellular molecules detected on a surface of graphene layer that alters electrical current through the graphene layer sensing concentrations of the pre-determined intracellular molecules in the pre-determined biological tissue, and
a silicon carbide (SiC) coating, including an inert coating biocompatible for a plurality of different pre-determined types of biological tissue, configured for coating and protecting the photovoltaic panel (PV) layer, QD-LED layer, logic layer and FET layer of the plurality of nanofid apparatus within the pre-determined biological tissue,
wherein the plurality of the nanofid apparatus are inserted directly into a plurality of individual cells in the pre-determined biological tissue for sensing concentrations of the pre-determined intracellular molecules in the plurality of individual cells in the pre-determined biological tissue, without causing any harm, toxicity or death to the plurality of individual cells in the pre-determined biological tissue or the pre-determined biological tissue;
the NIR light source apparatus external to the plurality of nanofid apparatus, configured for providing NIR light at a plurality of different wavelengths via optical fibers external to the plurality of nanofid apparatus;
an nanofid optical collection system external to the plurality nanofid apparatus, configured for collecting output NIR light data from the NIR light at the plurality of different wavelengths emitted from the plurality of QD-LEDs on the plurality of nanofid apparatus inserted into the plurality of individual cells in the pre-determined biological tissue, the output NIR light data received from the plurality of QD-LEDs activated by the FET layer and the molecule sensing layer sensing concentrations of the pre-determined intracellular molecules in the plurality of individual cells in the pre-determined biological tissue;
a nanofid signal processing system external to the plurality of nanofid apparatus, configured for processing the output NIR light data collected by the optical collection system with one or more signal processing methods and creating signal processing data with the one or more signal processing methods;
a nanofid data processing system external to the plurality of nanofid apparatus, configured for analyzing the created signal processing data from the signal processing system and producing and displaying an output report for sensing concentrations of the pre-determined intracellular molecules in the plurality of individual cells in the pre-determined biological tissue sensed by the plurality of nanofid apparatus inserted into the plurality of individual cells in the pre-determined biological tissue; and
a nanofid injection system external to the plurality of nanofid apparatus, including an injection apparatus and an injection process configured for injecting the plurality of nanofids apparatus directly into the plurality of individual cells in the pre-determined biological tissue without causing any harm, toxicity or death to the plurality of individual cells in the pre-determined biological tissue or the pre-determined biological tissue during the injection process.

17. The nanofid apparatus of claim 16, wherein the nanofid signal processing system includes one or more signal processing methods comprising a strobed input coding method comprising:
strobing a plurality of PV layers in a plurality of nanofid apparatus in a plurality of individual cells in a pre-determined tissue with NIR light pulse from an external NIR light source using a pre-determined time interval to provide power to the plurality of nanofid apparatus in the plurality of individual cells in the pre-determined tissue;
activating via the plurality of strobed PV layers a plurality of logic layers and a plurality of graphene FET layers and a plurality molecule sensing layers in the plurality of nanofid apparatus in the plurality of individual cells;
sensing in the plurality of graphene FET layers and the plurality of molecule sensing layers in the plurality of nanofid apparatus in the plurality of different individual cells for in changes concentrations in the pre-determined molecule of interest and generating voltage pulses to a plurality of logic layers in the plurality of nanofid apparatus when the changes concentrations in the pre-determined molecule of interest are detected;
determining whether on a pulse comparator in a logic layer in a nanofid apparatus whether a current increase between the two strobed pulses that exceeds a pre-determined threshold value, and if so
generating strobed input coding with light pulses of different pre-determined wavelength using bandpass filters to strobe and activate different sets of nanofid apparatus in different sets of cells using the light pulses of the different pre-determined wavelengths,
the strobed input coding activating a plurality of desired QD-LED tiles in the in the plurality of QD-LED layers on the plurality of nanofid apparatus;
emitting from the plurality of desired QD-LED tiles in the plurality of QD-LED layers on the plurality of nanofid apparatus when strobed with strobed input coding one of about thirty different NIR light output wavelengths,
each of the plurality of QD-LED tiles emitting a different NIR output wavelength, and a plurality of different QD-LED tiles in the plurality of QD-LED layers on the plurality of nanofid is selected by the plurality of pulse comparators for each time the plurality of QD-LED layers are activated in the plurality of nanofid apparatus for the plurality of individual cells; and
emitting light scatter from the plurality of nanofid apparatus in the plurality of individual cells with a unique NIR identifier for each individual cell in the plurality of cells for detection by a single optical fiber in the optical fiber array in the nanofid optical collection system.

18. The nanofid apparatus system of claim 16, wherein the nanofid optical collection system includes: a plurality of optical fibers, one or more optical fiber arrays including the plurality of optical fibers, an analog-to-digital converter (ADC) for sending the plurality of output NIR light data at a plurality of different wavelengths emitted the plurality of nanofid apparatus inserted into the plurality of individual cells in the pre-determined biological tissue to a Fiber Bragg Grating (FBG) system used to sort the plurality of NIR light data at the plurality of different wavelengths into distinct discrete bins, the FBG comprising: a plurality of FBG grates, a laser component, an optical beam splitter component, a plurality of optical circulators, one or more optical amplifiers, a spectrometer and one or more digital signal processors (DSP), wherein FBG system receives light scatter information from the optical fiber array and collects a plurality of unique NIR identifiers from the plurality of nanofid apparatus inserted into the plurality of individual cells in the pre-determined biological tissue for further processing by applications in the nanofid data processing system.

19. A method for determining intracellular activity with nanofid apparatus, comprising:
providing a plurality of nanofid apparatus that are completely inserted into a plurality of cells, the plural nanofid apparatus including a plurality of functional layers for detecting a biological substance of interest in the plurality of cells;
providing a nanofid optical collection system with an optical fiber array external to the plurality of nanofid apparatus;
providing a Near Infrared (NIR) light source external to the plurality of nanofid apparatus to provide a power source to the plurality of nanofid apparatus in the plurality of cells with NIR light;
providing one or more signal processing methods to process inputs and outputs from the plurality of nanofid apparatus in the plurality of cells, the one or more signal processing methods including processing inputs made from the NIR light source to activate the plurality of nanofid apparatus in the plurality of cells and processing outputs with emissions of NIR light from the plurality of nanofid apparatus including a plurality of unique cell identifiers when the biological substance of interest is detected in the plurality of cells with the nanofid optical collection system;
inserting the plurality of nanofid apparatus inside the plurality of cells;
detecting the biological substance of interest in the plurality of cells with the plurality of nanofid apparatus in the plurality of cells with the nanofid optical collection system with the optical fiber array external to the plurality of nanofid apparatus and the one or more signal processing methods;
collecting information about the biological substance of interest in the plurality of cells with the plurality of nanofid apparatus with the nanofid optical collection system with the optical fiber array and one or more different hardware devices external to the plural nanofid apparatus and the one or more signal processing methods;
processing the collected information uniquely identifying and recording all cell activity for the biological substance of interest in the plurality of cells to the individual cell level with a plurality of different hardware devices to create digital cell activity information about the plurality of cells;
processing the collected digital cell activity information from the one or more different hardware devices uniquely identifying and recording all cell activity for the biological substance of interest in the plurality of cells to the individual cell level with one or more applications on one or more network devices, each with one or more processors to create an output report; and
displaying on the output report on the one or more applications on the one or more network devices uniquely identifying and recording all cell activity for the biological substance of interest in the plurality of cells to the individual cell level.

20. A method for determining intracellular activity with nanofid apparatus, comprising:
injecting a plurality of nanofid apparatus into a plurality of individual cells in a pre-determined tissue type with a nanofid injection system;
applying input Near InfraRed (NIR) light from a NIR light source apparatus external to the plurality of nanofid apparatus, via a plurality of optical fibers external to the plurality of nanofid apparatus to provide power and activate the plurality of nanofid apparatus in the plurality of cells in the pre-determined tissue type;
collecting on a nanofid optical collection system external to the plurality nanofid apparatus output NIR light data at a plurality of different wavelengths emitted the plurality of nanofid apparatus inserted into the plurality of individual cells in the pre-determined biological tissue,
the output NIR light data received from the plurality of nanofid apparatus sensing concentrations of pre-determined intracellular molecules in the plurality of individual cells in the pre-determined biological tissue;
processing on a nanofid signal processing system external to the plurality of nanofid apparatus to output NIR light data collected by the optical collection system with one or more signal processing methods and creating signal processing data with one or more signal processing methods;
creating on a nanofid data processing system external to the plurality of nanofid apparatus an output report from the created signal processing data created by the nanofid signal processing system from sensing concentrations of the pre-determined intracellular molecules in the plurality of individual cells in the pre-determined biological tissue sensed by the plurality of nanofid apparatus inserted into the plurality of individual cells in the pre-determined biological tissue; and
displaying on the nanofid data processing system external to the plurality of nanofid apparatus the created output report created from the created signal processing data created by the nanofid signal processing system from sensing concentrations of the pre-determined intracellular molecules in the plurality of individual cells in the pre-determined biological tissue sensed by the plurality of nanofid apparatus inserted into the plurality of individual cells in the pre-determined biological tissue.
